(19) United States
(12) Patent Application Publication
(10) Pub. No.: US 2011/0151455 A1
(43) Pub. Date: Jun. 23, 2011

(54) Fish-ribosyn for antibiotic susceptibility testing

(75) Inventors: Peter George Stroot, Lutz, FL (US); Samuel James Dupont, JR., Tampa, FL (US)

(73) Assignee: University of South Florida, Tampa, FL (US)

(21) Appl. No.: 12/807,036

(22) Filed: Aug. 25, 2010

Related U.S. Application Data
(63) Continuation-in-part of application No. 12/806,341, filed on Aug. 10, 2010.
Application No. 12/806,341 is a continuation of application No. 11/821,946, filed on Jun. 25, 2007, now Pat. No. 7,771,941.
(60) Provisional application No. 60/815,997, filed on Jun. 23, 2006.
(60) Provisional application No. 61/275,070, filed on Aug. 25, 2009.

(51) Int. Cl. C12Q 1/68; C12Q 1/18

(52) U.S. Cl. 435/6.11; 435/6.15; 435/32

(57) ABSTRACT

The subject invention concerns materials and methods for evaluating the susceptibility of bacterial cells to an antibiotic or other antimicrobial compound or agent. In one embodiment, a sample comprising a microbial population is exposed to an antibiotic of interest. The sample is then processed using FISH-RiboSyn methods to determine the specific growth rate of the antibiotic-exposed microbes as compared to an untreated control. The subject invention also concerns materials and methods for determining the most suitable and/or effective antibacterial treatment for a person or animal having a bacterial infection.

### CROSS-REFERENCE TO RELATED APPLICATION

The present application is a continuation-in-part of U.S. application Ser. No. 12/806,341, filed Aug.10, 2010, which is a continuation of U.S. application Ser. No. 11/821,946, filed Jun. 25, 2007, which claims the benefit of U.S. Provisional Application Ser. No. 60/815,997, filed Jun. 23, 2006, each of which is hereby incorporated by reference herein in its entirety, including any figures, tables, nucleic acid sequences, amino acid sequences, and drawings, and the present application also claims the benefit of U.S. Provisional Application Ser. No. 61/275,070, filed Aug. 25, 2009, which is hereby incorporated by reference herein in its entirety, including any figures, tables, nucleic acid sequences, amino acid sequences, and drawings.

### BACKGROUND OF THE INVENTION

Empiric antibiotic therapy has long been the standard approach for patient care and has demonstrated a significant reduction in patient mortality due to bacterial infection (Leibovici (1998); MacArthur et al. (2004)). According to a 2006 report from the United States Center for Disease Control (CDC), bacterial infections are still considered a major cause of death in the United States (Heron et al. (2009)). Bacterial and viral infections that cause pneumonia and influenza were ranked eighth, while septicemia was ranked tenth. This is a dramatic improvement over the historically high percentage of death related illnesses attributed to bacterial infection before the use of empiric antibiotic therapy strategies (CDC (1999)). In general, empiric antibiotic therapy relies on treatment of the patient's infection with a broad spectrum antibiotic, while a comprehensive analysis of the pathogenic bacteria is completed in the clinical microbiology laboratory. This analysis includes the identification of the pathogenic bacteria and antibiotic susceptibility testing, both of which typically rely on use traditional culture based methods. If resistance to the broad spectrum antibiotic is revealed by this analysis, the physician will switch treatment, and deliver an appropriate antibiotic. While this antibiotic treatment strategy has been highly successful, the emergence of multiply resistant infections renders broad spectrum antibiotics less effective, and in general, has led to an increase in patient mortality (Zaragoza et al. (2003)). Furthermore, the improper use of antibiotics has been repeatedly identified as a primary cause of the proliferation of antibiotic resistance in infectious bacteria, and in some cases, can cause the patient to become a long term carrier of antibiotic resistant bacteria (Levy (1998); Sjolund et al. (2003)). A recent survey released by the CDC, CDC Foundation, and Amgen highlights infection as an emerging problem in the successful treatment of cancer patients, with the greatest concern focused on the proliferation of antibiotic resistant infections (Amgen, (2009)). Cancer patients undergoing chemotherapy are at great risk of infection due to the compromised state of their immune system. Risk of infection and inappropriate infection treatment present deadly obstacles to uninterrupted and effective chemotherapy and often result in elevated health care costs and an increased rate of patient mortality.

There has been substantial global concern over the emergence of multidrug-resistant strains of the non-motile gram-negative bacterium Acinetobacter baumannii, which has been commonly isolated in heath care environments with a propensity to infect immunosuppressed individuals (Perez et al. (2007)). Recently, an extensively resistant strain of A. baumannii has been isolated from an intensive care unit in the University of Pittsburgh Medical Center (Doi et al. (2009)). This strain of A. baumannii showed no susceptibility to any of the commercially available antibiotics and represents the growing concern of potential outbreaks of untreatable infections. Multiple antibiotic resistant strains of A. baumannii also threaten men and women serving in the military, especially in remote regions such as Iraq (Davis et al. (2005); Schafer and Mangino (2008)). Additionally, concern arises when transport of these organisms from the military theater of operation to hospitals, via an infected patient, complicates the already difficult battle of secondary infection in hospitals (Jones et al. (2006)).

The emergence of multiply drug-resistant (MDR) bacteria has become of critical concern in the treatment of infections in clinical settings. Additionally, it has shifted the mentality of antibiotic selection for patient treatment in an attempt to retard further proliferation of resistance against popular broad spectrum antibiotics. Kollef et al. designed a study to determine the relationship between hospital mortality and appropriate antimicrobial therapy (Kollef et al. (1999)). The study evaluated 2,000 patients requiring admittance to a medical or surgical ICU unit. The leading cause of inadequate antimicrobial therapy was the presence of both Gram-positive and Gram-negative antibiotic resistant infections which led to a risk of hospital mortality four times greater than properly treated patients.

Multiple antibiotic resistances are increasing in prevalence and severity at an alarming rate across the globe (Jones et al. (2008)). This, combined with newly emerging infectious diseases caused by bacteria with unknown antibiotic susceptibilities, present a significant burden on health care systems and the general public health. As resistance continues to outpace the drug discovery process, it is equally critical that new antibiotics targeted at multidrug-resistant pathogens be used judiciously in order to preserve their clinical utility. Judicious use requires knowing which antibiotics will actually work against the bacteria in a specific clinical setting. In light of this, an inexpensive, rapid assay designed to determine the most effective antibiotic for a given infection is essential and could reduce the spread of antibiotic resistance by avoiding the use of ineffective antibiotics. An ideal approach would utilize the speed and accuracy of molecular biology based methods to detect the antibiotic-induced reduction in the generation of informational biomolecules, such as rRNA, that are directly linked to the growth of bacteria.

Previously, Cutter and Stroot described a new method, RT-RiboSyn, for measuring the specific growth rate, μ, of a defined organism type by using a reverse transcription technique designed to compare the cellular concentration of precursor 16S rRNA (pre-16S rRNA) to mature 16S rRNA over time for cells treated with the antibiotic chloramphenicol (Cutter and Stroot (2008)). Although RT-RiboSyn has extensive potential for investigating the microbial ecology of natural and engineered systems, its lengthy protocol, equipment requirements, and high level of technical skill make it unattractive for use in a clinical setting.

Historically, the mature 16S rRNA molecule has been used as the target of choice for identifying and enumerating cells from a phylogenetically distinct microbial population due to the large availability of target sites within each cell and its diversity of conserved and organism specific sequence information (Woese (1987)). Like the mature 16S rRNA, the pre-16S rRNA also has unique sequence information that can be used for identification. In addition, the pre-16S rRNA is an intermediate in ribosome synthesis for all bacteria and the indirect measurement of the cellular level of pre-16S rRNA can provide useful rate of ribosome synthesis information, and therefore specific growth rate.

For growing cells treated with chloramphenicol, cells accumulate precursor rRNA as a result of the cessation of ribosome synthesis due to inhibition of precursor rRNA maturation (Forget and Jordan (1970); Pace (1973)). Previous work demonstrated the capability of DNA probe sandwich hybridization assays in tandem with either chloramphenicol or rifampin to observe cellular changes in precursor rRNA in starved cells (Cangelosi and Brabant (1997)). However, because of lengthy and labor-intensive protocols and expensive supplies and analysis the aforementioned methods are not suitable for antibiotic susceptibility testing of clinical samples. Cutter and Stroot recently developed methodology to determine the specific rate of ribosome synthesis by measuring the accumulation of pre-16S rRNA relative to mature 16S rRNA in chloramphenicol treated cells through use of RT-RiboSyn, a novel reverse transcription and primer extension method (Cutter and Stroot (2008)). This approach yields the specific growth rate of the targeted population, as opposed to growth response or growth state, resulting in a more advanced method for the comprehensive evaluation of phylogenetically distinct microbial populations.

FISH has been increasingly utilized for identification and enumeration of phylogenetically distinct microbial populations in numerous sample types, including clinical. Since the recent FDA approval of FISH for routine use at the clinical level, it makes for a practical starting point for the development of a method designed to expedite antibiotic susceptibility testing (AdvanDx, (2009)). Early in the development of FISH for the investigation of microbial ecology, it was noted that information about cellular physiological state may be possible (DeLong et al. (1989)). When FISH is conducted with a fluorescently labeled probe that is designed to complement a unique site on the 16S rRNA and pre-16S rRNA of a targeted bacterial population, it has been shown that fluorescence intensity increases as the specific growth rate of the cells increases (i.e., faster growing cells have higher numbers of ribosomes) (Dennis and Bremer (1974); Koch (1971); Waldron and Lacroute (1975)). This proportionality has been used to estimate the specific growth rate of individual cells of a targeted bacterial population in biofilms, however it is unable to distinguish between actively growing cells, cells in early stationary phase, and cells entering log-growth phase (Poulsen et al. (1993)).

Fluorescence in situ hybridization (FISH) is gaining in acceptance in clinical microbiology laboratories for the detection and identification of pathogenic bacteria in samples collected from patients. The FISH method relies on the use of fluorescently labeled probes that target specific sites on the 16S rRNA, which are unique to a specific organism type or group (DeLong et al. (1989)). The strength of the FISH method over other conventional identification techniques is in its inexpensive and rapid assay without the need for specialized technicians or cumbersome and expensive lab equipment. A FISH method employing a proprietary peptide nucleic acid based probe (PNA FISH) was recently used to identify A. baumannii in a positive blood culture sample demonstrating the potential use of a FISH based method designed to compliment current traditional clinical evaluation of positive blood cultures (Kempf et al. (2000); Peleg et al. (2009)). Recently, a PNA FISH based protocol for detection of Escherichia coli, Klebsiella pneumoniae and Pseudomonas aeruginosa in positive blood culture, developed by AdvanDx, has been approved by the U.S. Food and Drug Administration for use in clinical laboratories (AdvanDx, (2009)). The approval of this product is significant and shows a shift towards the use of molecular biology tools, such as FISH based methods, for rapid assessment of the proper therapeutic strategies in clinical settings. Cutter and Stroot have developed FISH-RiboSyn, a new method that uses FISH for measuring the rate of ribosome synthesis in a targeted bacterial population. This new method is an extension of the RT-RiboSyn method of Cutter and Stroot for the measurement of the rate of ribosome synthesis in Acinetobacter species (Cutter and Stroot (2008)).

### BRIEF SUMMARY OF THE INVENTION

The subject invention concerns materials and methods for evaluating the susceptibility of bacterial cells to an antibiotic or other antimicrobial compound or agent. In one embodiment, a sample comprising a microbial population is exposed to an antibiotic of interest. The sample is then processed using FISH-RiboSyn methods to determine the specific growth rate of the antibiotic-exposed microbes as compared to an untreated control. In an exemplified embodiment, FISH-RiboSyn was employed to evaluate the antibiotic susceptibility of A. baumannii strains with differing susceptibility to two antibiotics: doxycycline, a bactericidal protein synthesis inhibitor and levofloxacin, a bacteriostatic DNA synthesis inhibitor.

The subject invention also concerns materials and methods for determining the most suitable and/or effective antibacterial treatment for a person or animal having a bacterial infection.

FISH-RiboSyn was developed for the estimation of the specific growth rate, μ, of a distinct microbial population. This method measures the rate of ribosome synthesis in a target population by using fluorescence in situ hybridization (FISH). This approach combines the phylogenic specificity associated with FISH and the inhibition of secondary ribosome processing by the antibiotic chloramphenicol to measure the accumulation of precursor 16S rRNA as a function of chloramphenicol exposure time. This data was then compared to the measurement of μ for a pure culture using spectrophotometry. FISH-RiboSyn was tested on three pure cultures of Acinetobacter spp. grown at different μ. Each species showed a defined increase in fluorescence intensity over a period of chloramphenicol exposure demonstrating the effectiveness of this method. The relationship between the slope of the time based increase in mean whole cell fluorescence and μ was linear and independent of the three species. The FISH-RiboSyn method was then used to evaluate the impact of two antibiotics, doxycycline and levofloxacin, on the rate of ribosome synthesis of a susceptible and resistant strain of A. baumannii. A distinct difference between impacted and resistant cells was observed for these organisms and demonstrates the potential use of FISH-RiboSyn as a rapid and inexpensive screening procedure for patients with bacterial infections.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C: Plot of the mean whole cell fluorescence (F) for a pure culture of A. calcoaceticus^{T} as a function of chloramphenicol exposure time (t_{cm}) when grown at 30° C. (μ=1.18 hr⁻¹) in nutrient broth alongside representative images of the observed increase in F after chloramphenicol exposure times of 0 (FIG. 1A), 10 (FIG. 1B), and 20 (FIG. 1C) minutes. Error bars represent one standard deviation and size bar represents 10 μm.

FIG. 2: Plot of dF/dt_{cm} calculated by image analysis of chloramphenicol treated cells as a function of specific growth rate (μ) as determined by OD measurements for A. calcoaceticus^{T} (□), A. baumannii^{T} (⋄), and A. lwoffii^{T} (∘).

FIGS. 3A-3D: Plot of OD vs. time with secondary y-axis representing the ribosome synthesis rate (dF/dt_{cm}) for doxycycline treated A. baumannii^{T} and control (FIG. 3A) and A. baumannii CBD1311 and control (FIG. 3B) as well as levofloxacin treated A. baumannii^{T} and control (FIG. 3C) and A. baumannii CBD1311 and control (FIG. 3D). The represents OD of antibiotic treated culture, □ represents dF/dt of antibiotic treated culture, ⋄ (filled) represents OD of control culture, and ⋄ represents dF/dt of control culture. Arrows denote endpoint of constant log-growth.

FIGS. 4A-4D: Plot of mean whole cell fluorescence (F) vs. chloramphenicol exposure time (t_{cm}) for (FIG. 4A) A. baumannii^{T} treated with doxycycline (□) after 30 minutes of exposure and its control (Δ), (FIG. 4B) A. baumannii CBD 1311 treated with doxycycline (□) after 30 minutes of exposure and its control (Δ), (FIG. 4C) A. baumannii^{T} treated with levofloxacin (□) after 90 minutes of exposure and its control (Δ), (FIG. 4D) A. baumannii CBD 1311 treated with doxycycline (□) after 90 minutes of exposure and its control (Δ).

FIGS. 5A-1 through 5D-3: Representative images of A. baumannii^{T} treated with doxycycline (FIGS. 5A-1, 5A-2, and 5A-3) and the appropriate control (FIGS. 5B-1, 5B-2, and 5B-3) after 30 minutes of exposure and representative images of A. baumannii^{T} treated with levofloxacin (FIGS. 5C-1, 5C-2, and 5C-3) and the appropriate control (FIGS. 5D-1, 5D-2, and 5D-3) after 90 minutes of exposure. In each series images 1, 2, and 3 represent 0, 10, and 20 minutes of chloramphenicol exposure. Size bar=10 μm.

FIGS. 6A-1 through 6C-3: Histograms of levofloxacin treated A. baumannii^{T} cells after 30 (FIGS. 6A-1, 6A-2, and 6A-3), 90 (FIGS. 6B-1, 6B-2, and 6B-3), and 150 (FIGS. 6C-1, 6C-2, and 6C-3) minutes of exposure to the antibiotic and after 0 (1), 10 (2), and 20 (3) minutes of exposure to chloramphenicol.

FIGS. 7A-7C: FISH images of A. lwoffii cells exposed to chloramphenicol for 0 (FIG. 7A), 10 (FIG. 7B), and 20 (FIG. 7C) minutes. Cells were collected from the mother culture at the same time antibiotics were administered to the experimental cultures.

FIG. 8: dF/dt for untreated A. lwoffii at 30, 90, and 210 minutes after transfer to fresh media. Note that time 0 represents cells taken directly from the mother culture. Error bars represent 1 standard deviation.

FIG. 9: Images taken at various chloramphenicol exposure times (y-axis) for ampicillin exposure times of 30 (top row) and 150 (bottom row) minutes to demonstrate the reduction in dF/dt.

FIG. 10: dF/dt for Ampicillin treated A. lwoffii at 30, 90, 150 and 210 minutes after transfer to fresh media. Error bars represent 1 standard deviation.

FIG. 11: Images taken at various chloramphenicol exposure times (y-axis) for Ciprofloxacin exposure times of 30 (top row) and 210 (bottom row) minutes to demonstrate the reduction in dF/dt.

FIG. 12: dF/dt for Ciprofloxacin treated A. lwoffii at 30, 90, 150 and 210 minutes after transfer to fresh media. Error bars represent 1 standard deviation.

FIG. 13: Images taken at various chloramphenicol exposure times (y-axis) for cells treated with Doxycycline for 30 minutes. Images taken with DAPI (top row) and Cy3 (bottom row).

FIG. 14: dF/dt for Doxycycline treated A. lwoffii at 30 minutes after transfer to fresh media. Error bars represent 1 standard deviation.

FIG. 15: Optical density readings for the mother culture and the subsequent experimental cultures. The yellow boxed data point on the mother culture curve represents the point at which subcultures were generated.

FIG. 16: Optical density and dF/dt for control and experimental batch cultures.

### BRIEF DESCRIPTION OF THE SEQUENCES

SEQ ID NO:1 is an oligonucleotide hybridization probe that can be used according to the present invention.

### DETAILED DESCRIPTION OF THE INVENTION

The subject invention concerns methods for determining the effect of an antimicrobial compound on specific growth rate of a microbial population. In one embodiment, a sample comprising a microbial population is exposed to an antimicrobial compound of interest. The sample is then processed using FISH-RiboSyn methods to determine the specific growth rate of the antimicrobial compound-exposed microbes. FISH-RiboSyn methods are described in published U.S. Patent Application No. US-2008-0009011.

In one embodiment, a method of the invention comprises:

1) exposing a sample comprising microbes to an antimicrobial compound for a period of time;

2) exposing the sample to a protein synthesis inhibitor (e.g., chloramphenicol);

3) collecting a time series of samples (preferably at defined times) from step 2; and

4) analyzing the collected samples to measure the rate of precursor rRNA buildup in the microbes and determining the effect of the antimicrobial compound on the specific growth rate of the microbes. In one embodiment, the collected samples are analyzed in comparison to an untreated control.

In one embodiment, the rate of precursor rRNA buildup is measured in situ using FISH. The rate of precursor rRNA accumulation relative to mature rRNA is indicative of the specific growth rate of the microbes being exposed to the antimicrobial compound. In a specific embodiment, the precursor rRNA is prel 6S rRNA.

The subject invention also concerns materials and methods for determining the most suitable and/or effective antimicrobial treatment for a person or animal having a microbial infection. In one embodiment, a method comprises obtaining a sample comprising microbes from the person or animal and then exposing the sample to an antimicrobial compound to be tested. The sample is then analyzed using a FISH-RiboSyn method of the invention to determine the susceptibility of the microbes to the antimicrobial compound. Based on the results, a determination is then made as to whether the antimicrobial compound is suitable and/or effective for treating the infection in the person or animal. Criteria for deciding whether an antimicrobial compound is suitable and/or effective for treating the infection can readily be determined by a clinician of ordinary skill in the art. The method can also optionally comprise treating the person or animal with the antimicrobial compound if it is determined that the compound is a suitable and/or effective treatment against the microbe infecting the person or animal. The method can be used to assay one or more antimicrobial compounds, or even a combination of one or more antimicrobial compounds, for suitability and/or effectiveness in treating an infection. In one embodiment, the method is performed multiple times over the course of treatment of the person or animal to monitor that the compound retains effectiveness against the microbe infecting the person or animal.

The specific growth rate (or cell doubling time) for a distinct microbial population can be determined and, optionally, monitored by its rate of precursor rRNA buildup. Distinct microbial populations can be targeted exclusively by using oligonucleotide probes or primers that target signature sequence information within the precursor 16S rRNA or mature 16S rRNA.

In one embodiment, a method of the invention measures the increase of pre16S rRNA in individual cells of a specific microbial population. FISH-RiboSyn is an in situ method that utilizes fluorescence in situ hybridization (FISH) with specific probes or primers that target: (1) 5′ or 3′ end of pre16S rRNA or (2) the interior region of both pre16S rRNA and mature 16S rRNA. Images are captured at defined exposure times and the average fluorescent intensity for individual cells can be determined. These intensities are used to calculate the rate of increase of the prel 6S rRNA. When a sample is exposed to chloramphenicol or other protein synthesis inhibitor for defined times, the rate of increase of the prel 6S is determined and the specific growth rate is calculated.

Optionally, in the various embodiments of the invention, the method further comprises recording the determined specific growth rate or specific rate of ribosome synthesis of a rapidly growing cell population in physical or electronic media. Preferably, the specific rate of ribosome synthesis and/or the specific growth rate are recorded or otherwise stored as units of synthesis or growth per unit of time. Optionally, the recorded growth or synthesis rate includes an annotation conveying the growth conditions (e.g., culture conditions) under which the determination was made, such as temperature. In one embodiment, the rate of pre16S rRNA buildup relative to the 16S rRNA is measured and input into a computer algorithm that then calculates the specific rate of ribosome synthesis. Optionally, the specific growth rate or the specific rate of ribosome synthesis can be displayed on an output device, such as an analog recorder, teletype machine, typewriter, facsimile recorder, cathode ray tube display, computer monitor, or other computation device. Optionally, the displayed specific growth rate or specific ribosome synthesis rate includes an annotation conveying the growth conditions (e.g., culture conditions) under which the determination was made (such as temperature).

Optionally, in the various embodiments of the invention, the method further comprises carrying out a manipulation of the non-homogeneous system based on the determined specific growth rate or specific ribosome synthesis rate. The manipulation can comprise, for example, a modification of culture conditions or the provision of a signal to induce expression of a polynucleotide of interest by one or more microbial populations within the system. In one embodiment, the manipulation comprises the addition of a substance that alters the metabolic rate of the one or more populations of microbes within the system. For example, the manipulation may comprise the addition of supplements such as carbon, nitrogen, and/or inorganic phosphates, or modification of temperature and/or pH.

Optionally, in the various embodiments of the invention, the method further comprises comparing the specific growth rate of a cell population within the non-homogeneous system, as determined above, to pre-existing growth rate data characterizing cell populations, such as microbial organisms. The pre-existing growth rate data of a cell population may be that specific growth rate observed under particular growth conditions (e.g., culture conditions), such as at a given temperature or at a given cell number or cell density, for example.

Optionally, in the various embodiments of the invention, the method further comprises introducing a test agent to the non-homogeneous system, or a sample thereof, before, during, or after introduction of the protein synthesis inhibitor, in order to determine whether the test agent exerts a biological effect on the microbes. The test agent may be a member of a combinatorial library, for example. In one embodiment, the method includes contacting the non-homogeneous system, or a sample thereof, with one or more members of a library of agents for the purpose of monitoring the effect on specific growth rate. Optionally, the method further comprises comparing the specific growth rate of a particular microbial population within the non-homogeneous system before and after introduction of the test agent. The particular microbial population may be one that is determined to be rapidly growing in the presence or absence of the test agent, for example.

In the methods and kits of the invention, the probe and primer is preferably genus-specific, species-specific, or strain-specific. Reference herein to “primer” or “probe” is not to be taken as any limitation as to structure, size, or function. The primer may be used as an amplification molecule or may be used as a probe for hybridization purposes.

Another aspect of the invention concerns a kit for use in practicing the above method. The kit, in compartmental form, comprising one or more compartments or containers adapted to contain one or more antibiotics and one or more oligonucleotide probes or primers that target signature sequence information within the precursor rRNA or mature rRNA. In one embodiment, the probes and/or primers target sequences within the precursor 16S rRNA or mature 16S rRNA. Preferably, the primers are capable of participating in an amplification reaction of DNA comprising: (1) the 5′ or 3′ end of precursor 16S rRNA; or (2) the interior region of both precursor 16S rRNA and mature 16S rRNA. Preferably, the oligonucleotide probe targets: (1) the 5′ or 3′ end of precursor 16S rRNA; or (2) the interior region of both precursor 16S rRNA and mature 16S rRNA. Optionally, the kit contains another compartment or container adapted to contain reagents to conduct an amplification reaction. In one embodiment, the probe is labeled at its 5′ end by a fluorogenic reporter molecule and at its 3′ end by a molecule capable of quenching said fluorogenic molecule. In a specific embodiment, the probe is a fluorescently-labeled oligonucleotide hybridization probe targeting the precursor 16S rRNA for members of a selected genus, conjugated with a dye such as a cyanine dye.

As indicated above, kits of the invention include reagents for use in the methods described herein, in one or more containers. The kits may include antibiotics, primers and/or probes, buffers, and/or excipients, separately or in combination. Each reagent can be supplied in a solid form or liquid buffer that is suitable for inventory storage. Kits may also include means for obtaining a biological sample of a tissue or biological fluid from a host organism or an environmental sample.

Kits of the invention are provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe or primer. Preferably, the solid matrix is a planar material such as the side of a microtitre well or the side of a dipstick. In one embodiment, the kit includes a microtitre tray with two or more wells and with reagents including primers or probes in the wells.

The one or more probes or primers in the kit may be immobilized to the compartments. Methods for linking nucleic acid molecules to solid supports are well known in the art. Processes for linking the primer or probe to the solid matrix include amide linkage, amidate linkage, thioether linkage, and the introduction of amino groups on to the solid matrix. The kit may be conveniently adapted for automated or semi-automated use. The kit may include a plurality of primers and/or probes that target either the 5′ or 3′ end of prel 6S rRNA, or the interior region of both prel 6S rRNA and mature 16S rRNA, to permit the detection and determination of specific growth rate of more than one microbe. Optionally, the probes and primers are arrayed in the compartments of the kits.

Kits of the invention may optionally include a set of instructions in printed or electronic (e.g., magnetic or optical disk) form, relating information regarding the components of the kits and/or how to measure specific growth rate of a microbe. The kit may also be commercialized as part of a larger package that includes instrumentation for measuring other biochemical components, such as, for example, a mass spectrometer.

The sample may be a biological sample. In one embodiment, one or more biological samples can be obtained from an individual. The biological sample may be obtained by any method known in the art. Samples may be collected at a single time point or at multiple time points from one or more tissues or bodily fluids. The tissue or fluid may be collected using standard techniques in the art, such as, for example, tissue biopsy, blood draw, or collection of secretia or excretion from the body. Examples of suitable bodily fluids or tissues from which an infectious agent, or component thereof, may be isolated include urine, blood, intestinal fluid, edema fluid, saliva, lacrimal fluid (tears), inflammatory exudate, synovial fluid, abscess, empyema or other infected fluid, cerebrospinal fluid, pleural effusions, sweat, pulmonary secretions, seminal fluid, feces, bile, intestinal secretions, or any infected tissue including, but not limited to liver, intestinal epithelium, spleen, lung, pericardium, pleura, skin, muscle, synovium, cartilage, bone, bone marrow, thyroid gland, pancreas, brain, prostate, ovaries, endometrium, uterus, uterine cervix, testes, epididymis, bladder wall, kidney, adrenal, pituitary gland, adipose cells/tissue, omentum, or other cells and tissue. The frequency of obtaining one or more biological samples can vary.

Oligonucleotides can be of any suitable size, which depends on many factors, including the function or use of the oligonucleotide. Oligonucleotides can be prepared by any suitable method, including, for example, cloning, enzymatic restriction of larger nucleotides, and direct chemical synthesis by a method such as the phosphotriester method of Narang et al. (1979), the phosphodiester method of Brown et al. (1979), the diethylphosphoramidite method of Beaucage et al. (1981), and the solid support method of U.S. Pat. No. 4,458,066. A review of synthesis methods is provided in Goodchild (1990).

The term “primer” refers to an oligonucleotide, whether natural or synthetic, capable of acting as an initiating point for DNA synthesis under conditions in which synthesis of a primer extension product complementary to a nucleic acid strand is induced. For example, such conditions include inclusion of four different nucleoside triphosphates and an agent for polymerization (i.e., DNA polymerase or reverse transcriptase) in an appropriate buffer and at a suitable temperature. A primer can be a single-stranded oligodeoxyribonucleotide. The length of a primer can vary and depends on the intended use of the primer. In one embodiment, a primer is less than 40 nucleotides. In another embodiment, a primer ranges from 15 to 35 nucleotides.

A primer need not reflect the exact sequence of the template, but should be sufficiently complementary to hybridize with a template. Primers can incorporate additional features which allow for the detection or immobilization of the primer, but do not alter the basic ability of the primer to act as a point of initiation of DNA synthesis. The primers and oligonucleotide probes may be manufactured using any convenient method of synthesis. Examples of such methods may be found in standard textbooks, for example Agrawal (1993). The primers and probes can be produced by recombinant or synthetic techniques. If desired, the primer(s) may be labeled to facilitate detection.

The isolated polynucleotides (e.g., oligonucleotide detection probes and primers) used in the invention are capable of selectively hybridizing to a nucleic acid sequence of the precursor rRNA, such as precursor 16S rRNA (e.g., amplification of the 5′ or 3′ end of precursor 16S rRNA; or the interior region of both precursor 16S rRNA and mature 16S rRNA). An oligonucleotide probe will typically comprise a contiguous/consecutive span of at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 or more nucleotides. In one embodiment, the oligonucleotide probe is 17-50 nucleotides. In another embodiment, the oligonucleotide probe is 17-30 nucleotides. In another embodiment, the oligonucleotide probe is 17-30 nucleotides.

The design of such probes and primers will be apparent to a person of ordinary skill in the art. Typically, the oligonucleotide probe (also referred to herein as the “detection probe”, “sequence-specific probe”, or “precursor rRNA-specific probe”) comprises a recognition sequence that is partially or fully complementary to a target nucleic acid sequence (e.g., DNA or RNA), in this case, a nucleic acid sequence of a precursor rRNA, e.g., precursor 16S rRNA. Optionally, the recognition sequence is substituted with high-affinity nucleotide analogues to increase the sensitivity and/or specificity of conventional oligonucleotides, for hybridization to target sequences.

Such probes are of any convenient length such as up to 50 nucleotides, up to 40 nucleotides, and more conveniently up to 30 nucleotides in length, such as for example 8-25 or 8-15 nucleotides in length. In general, such probes will comprise base sequences entirely complementary to the corresponding locus of the target sequence. However, if required, one or more mismatches may be introduced, provided that the discriminatory power of the oligonucleotide probe is not unduly affected. The probes may carry one or more labels to facilitate detection.

The label of the labeled probes and primers can be any type of detectable substance, such as a radioactive label, enzyme label, chemiluminescent label, fluorescent label, or magnetic label. Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications.

In some embodiments, the oligonucleotide probe comprises a fluorophore moiety and a quencher moiety, positioned in such a way that the hybridized state of the probe can be distinguished from the unhybridized state of the probe by an increase in the fluorescent signal from the nucleotide. In one aspect, the detection probe comprises, in addition to the recognition sequence (also known as the recognition element), first and second complementary sequences, which specifically hybridize to each other when the probe is not hybridized to a recognition sequence in a target molecule, bringing the quencher molecule in sufficient proximity to the reporter molecule to quench fluorescence of the reporter molecule. Hybridization of the target sequence distances the quencher from the reporter molecule and results in a signal, which is proportional to the amount of hybridization.

As used herein, the term “label” includes a reporter group, which is detectable either by itself or as a part of a detection series. Examples of functional parts of reporter groups are biotin, digoxigenin, fluorescent groups (groups which are able to absorb electromagnetic radiation, e.g., light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-l-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g., substituted organic nitroxides) or other paramagnetic probes (e.g., Cu²⁺, Mg²⁺) bound to a biological molecule being detectable by the use of electron spin resonance spectroscopy). Particular examples of such labels are biotin, fluorescein, Texas Red, rhodamine, dinitrophenyl, digoxigenin, Ruthenium, Europium, cyanine dyes such as Cy5 and Cy3, etc. In one embodiment, the label is a dye, such as a cyanine dye, conjugated to the oligonucleotide probe (e.g., Cy3).

Preferably, the probe or primer specifically hybridizes with at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides of the target sequence (such as the 5′ or 3′ end of precursor 16S rRNA; or the interior region of both precursor 16S rRNA and mature 16S rRNA). Various degrees of stringency of hybridization can be employed. The more stringent the conditions, the greater the complementarity that is required for duplex formation. Stringency of conditions can be controlled by temperature, probe concentration, probe length, ionic strength, time, and the like. Preferably, hybridization is conducted under low, intermediate, or high stringency conditions by techniques well known in the art, as described, for example, in Keller and Manak (1987).

For example, hybridization of immobilized DNA on Southern blots with ³²P-labeled gene-specific probes can be performed using standard methods (Maniatis et al. (1982)). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6×SSPE, 5× Denhardt's solution, 0.1% SDS, 0.1 mg/ml denatured DNA. The melting temperature is described by the following formula (Beltz et al. (1983)).

Tm=81.5° C.+16.6 Log[Na⁺]+0.41(% G+C)−0.61(% formamide)−600/length of duplex in base pairs.

Washes are typically carried out as follows:
(1) twice at room temperature for 15 minutes in 1×SSPE, 0.1% SDS (low stringency wash);
(2) once at Tₘ −20° C. for 15 minutes in 0.2×SSPE, 0.1% SDS (intermediate stringency wash).

For oligonucleotide probes, hybridization can be carried out overnight at 10-20° C. below the melting temperature (Tₘ) of the hybrid in 6×SSPE, 5× Denhardt's solution, 0.1% SDS, 0.1 mg/ml denatured DNA. Tₘ for oligonucleotide probes can be determined by the following formula:

Tₘ(° C.)=2(number T/A base pairs)⁺4(number G/C base pairs) (Suggs et al. (1981)).

Washes can be carried out as follows:
(1) twice at room temperature for 15 minutes 1×SSPE, 0.1% SDS (low stringency wash);
2) once at the hybridization temperature for 15 minutes in 1×SSPE, 0.1% SDS (intermediate stringency wash).

In general, salt and/or temperature can be altered to change stringency. With a labeled DNA fragment >70 or so bases in length, the following conditions can be used:

Low: 1 or 2×SSPE, room temperature

Low: 1 or 2×SSPE, 42° C.

Intermediate: 0.2×or 1×SSPE, 65° C.

High: 0.1×SSPE, 65° C.

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al. (1989) and Ausubel et al. (1989).

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al. (1989), and Ausubel et al. (1989).

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein the mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense sequence”.

The term “label”, as used herein, refers to any atom or molecule that can be used to provide a detectable (preferably, quantifiable) signal, and which can be attached to a nucleic acid or protein. Labels may provide signals detectable by fluorescence, radioactivity, colorimetric, X-ray diffraction or absorption, magnetism, enzymatic activity, and the like.

The terms “recombinant host cells”, “host cells”, “cells”, “cell lines”, “cell cultures”, and other such terms refer to prokaryotic or eukaryotic cells which can be, or have been, used as recipients for recombinant vectors or other transfer DNA, immaterial of the method by which the DNA is introduced into the cell or the subsequent disposition of the cell. Thus, the cells subjected to the method of the invention can be, for example, any bacterial cells (e.g., Gram-positive, Gram-negative, those not lending themselves to Gram stain, aerobic, anaerobic, etc.), yeast cells, vertebrate cells (such as human or non-human mammalian cells), invertebrate cells, etc. The terms include the progeny of the original cell that has been transfected. The term “recombinant” when used with reference to a cell, or polynucleotide, polypeptide, or vector, indicates that the cell, polynucleotide, polypeptide or vector, has been modified by the introduction of a heterologous nucleic acid or amino acid or the alteration of a native nucleic acid or amino acid, or that the cell is derived from a cell so modified. A polypeptide of interest can be encoded by a gene that is part of the cell's genome, but for which regulatory sequences have been modified to provide increased levels of expression. Thus, recombinant cells can express genes that are not found within the native (non-recombinant) form of the cell or express native genes that are otherwise abnormally expressed, under expressed or not expressed at all. The prokaryotic or eukaryotic cells subjected to the method of the invention may be recombinant cells, un-modified cells, or a mixture thereof.

The term “genetic modification” as used herein refers to the stable or transient alteration of the genotype of a cell by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides. A translation initiation codon can be inserted as necessary, making methionine the first amino acid in the sequence. The terms “transfection” and “transformation” are used interchangeably herein to refer to the insertion of an exogenous polynucleotide into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

Examples of microorganisms that may be assayed for cell growth rate in accordance with the methods of the invention include, but are not limited, to those of importance in infections of humans and animals, wastewater and waste treatment processes (e.g., nitrifying bacteria, phosphorus accumulating organisms, and methanogens), public health (e.g., coliforms and bioterrorism agents) and food safety (e.g., botulism). Examples of potential bacterial cells of interest include, but are not limited to, Nitrospira spp., Nitrosospira spp., Nitrobacter spp., Nitrosomonas spp., Clostridium spp., Bacillus spp. (such as Bacillus anthracis), methogenic archaea, coliforms (Enterobacteriaceae, such as E. coli), Staphylococcus spp., Salmonella spp., Streptococcus spp., Chlamydia spp., Brucella spp., Yersinia spp., Shigella spp., Neisseria spp., Haemophilus spp., Listeria spp., Klebsiella pneumoniae, Pseudomonas spp., Mycobacterium spp., Bordetella spp., Actinomycetes spp., Vibrionaceae spp., Treponema spp., Legionella spp., Mycoplasma spp., Rickettsiae spp., and Bacteroides spp.

The medium used to cultivate the microorganisms may be any conventional medium suitable for growing the populations of microorganisms in question and, optionally, obtaining expression of a gene of interest. Microorganisms can be grown under amenable culture conditions, i.e., appropriate conditions of temperature, pH, humidity, oxygen, and nutrient availability including carbon/energy sources. Suitable media are available from commercial suppliers or may be prepared according to published protocols (e.g., as described in catalogues of the American Type Culture Collection).

Gene products secreted from the microbial cell populations in the mixed culture or samples derived there from may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography, or the like.

The exposing (e.g., contacting) steps of the method of the invention can involve combining or mixing the non-homogeneous sample and the protein synthesis inhibitor, or the probe or primers, in a suitable receptacle, such as a reaction vessel, microvessel, tube, microtube, well, or other solid support. Samples, protein synthesis inhibitors, and/or probes or primers may be arrayed on a solid support, such as a multi-well plate. Likewise, the sampling and analyzing (determining) steps can take place in an arrayed format on a solid support, such as a multi-well plate. “Arraying” refers to the act of organizing or arranging members of a library (e.g., an array of different samples, an array of protein synthesis inhibitors, or an array of primers or probes that target signature sequence information within the precursor rRNA or mature rRNA, such as precursor 16S rRNA or mature 16S rRNA), or other collection, into a logical or physical array. Thus, an “array” refers to a physical or logical arrangement of, e.g., library members (e.g., mixed culture library members). A physical array can be any spatial format or physically gridded format in which physical manifestations of corresponding library members are arranged in an ordered manner, lending itself to combinatorial screening. For example, samples corresponding to individual or pooled members of a sample library can be arranged in a series of numbered rows and columns, e.g., on a multiwell plate. Similarly, sensors can be plated or otherwise deposited in microtitered, e.g., 96-well, 384-well, or-1536 well, plates (or trays). Optionally, the protein synthesis inhibitors, primers, and probes may be immobilized on the solid support with retention of function. Methods for linking nucleic acid molecules and proteins to solid supports are well known in the art. Processes for linking the primer or probe to the solid matrix include amide linkage, amidate linkage, thioether linkage, and the introduction of amino groups on to the solid matrix.

As used herein, the term “protein synthesis inhibitor” is intended to refer to bacteriostatic agents that inhibit the secondary processing of precursor rRNA, but do not inhibit the production of precursor rRNA. For example, chloramphenicol, lincomycin, and erythromycin, are ribosomally active antibiotics that block the formation of peptide bonds by binding at or near the aminoacyl tRNA binding site on the large ribosomal subunit. After some time, the previously synthesized peptidyl tRNA is released and hydrolyzed. The ribosomal subunits are then released from the mRNA and are free to rejoin other mRNA molecules to start another abortive cycle. This leads to a truncated version of the ribosome cycle. Thus, these drugs inhibit protein synthesis at the chain elongation step, leading to premature association of the active complex. As a result, when these antibiotics are withdrawn, many free ribosomes are present and ready to resume normal protein synthesis. This explains why the action of these drugs is reversible and why these antibiotics are bacteriostatic instead of bacteriocidal. The protein synthesis inhibitor may be one that inhibits the secondary processing of rRNA in prokaryotic cells, eukaryotic cells, or both cell types.

As used herein, the terms “non-homogeneous system”, “non-homogeneous sample”, “mixed system”, and “mixed sample” are interchangeable and refer to a mixture of two or more cell populations (such as microbial populations), e.g., a mixed culture sample. The non-homogeneous system or sample can be any composition of matter of interest, in any physical state (e.g., solid, liquid, semi-solid, vapor) and of any complexity, such as a biological sample (e.g., a bodily fluid, plant or seed material) or environmental sample (e.g., water, soil, slurry). Preferably, the sample is a fluid, such as a bodily fluid. The sample may be contained within a test tube, culture vessel, fermentation tank, multi-well plate, or any other container or supporting substrate. The sample can be, for example, a cell culture, human or animal tissue (such as flesh, blood, saliva, semen, vaginal secretion, urine, tears, perspiration, extracellular fluid, etc.), or an environmental sample, such as water, soil, or sludge. The sample can be a small-scale or large scale fermentation.

The “complexity” of a sample refers to the number of different microbial species that are present in the sample.

The terms “body fluid” and “bodily fluid”, as used herein, refer to a mixture of molecules obtained from a patient. Bodily fluids include, but are not limited to, exhaled breath, whole blood, blood plasma, urine, semen, saliva, lymph fluid, meningal fluid, amniotic fluid, glandular fluid, sputum, feces, sweat, mucous, and cerebrospinal fluid. Bodily fluid also includes experimentally separated fractions of all of the preceding solutions or mixtures containing homogenized solid material, such as feces, tissues, and biopsy samples.

Biological samples (samples of biological origin) includes those that are accessible from an organism through sampling by invasive means (e.g., surgery, open biopsy, endoscopic biopsy, and other procedures involving non-negligible risk) or by minimally invasive or non-invasive approaches (e.g., urine collection, blood drawing, needle aspiration, and other procedures involving minimal risk, discomfort or effort). The definition also includes samples that have been manipulated in any way after their procurement, such as by treatment with reagents, solubilization, or enrichment for certain components, such as proteins, organic metabolites, or microbes. The term “biological sample” also encompasses a clinical sample such as serum, plasma, other biological fluid, or tissue samples, and also includes cells in culture, cell supernatants and cell lysates.

As used herein, the terms “population” and “cell population” are intended to refer to a distinguishable group of eukaryotic or prokaryotic cells, such as a genus, species or strain of microorganism. A population can differ from other populations by phylogenetic profile or by some other detectable genotype and/or phenotype. Using a method of the invention, populations can be distinguished from each other based on specific growth rate and length heterogeneity of the pre rRNA RT&PE products. A population can comprise two or more sub-populations that differ from each other by some detectable genotype and/or phenotype. A non-homogeneous system such as a mixed culture can be so small as to comprise two populations or can be larger, e.g., 10¹² populations. In some embodiments, a mixed culture is between five and 20 different populations, as well as up to hundreds or thousands of different populations. Those skilled in the art can readily determine a suitable size and diversity of a population sufficient for a particular application.

The terms “microbe” and “microbial cell” are inclusive of all prokaryotic microorganisms with a protein synthesis pathway susceptible to suppression by the protein synthesis inhibitor utilized in accordance with the invention. The microbe may be pathogenic or non-pathogenic. The microbe may be an infectious agent, such as a clinically important infectious agent. Examples of infectious agents include, but are not limited to bacteria, protozoa, and parasites, and any organism capable of replicating in a host organism, whether extracellularly, intracellularly, or both. See, e.g., Kobayashi et al. (2002), which is incorporated herein by reference in its entirety. A “clinically important infectious agent” is an infectious agent, microbial infectious agent, invading microbe, microbe, bacteria, protozoa, parasite, etc. that causes or is associated with a disease or pathological disorder in an individual.

The term “ex vivo,” as used herein, refers to an environment outside of a patient. Accordingly, a sample of bodily fluid collected from a patient is an ex vivo sample of bodily fluid as contemplated by the subject invention.

A “patient”, as used herein, refers to an organism, including mammals, from which samples can be collected in accordance with the present invention. Mammalian species that benefit from the disclosed systems and methods of detection include, and are not limited to, humans, apes, chimpanzees, orangutans, monkeys; and domesticated animals (e.g., pets) such as dogs, cats, mice, rats, guinea pigs, and hamsters.

“Monitoring” refers to recording changes in a continuously varying parameter, such as growth rate (e.g., doubling time).

A “solid support” (also referred to herein as a “solid substrate”) has a fixed organizational support matrix that preferably functions as an organization matrix, such as a microtiter tray. Solid support materials include, but are not limited to, glass, polacryloylmorpholide, silica, controlled pore glass (CPG), polystyrene, polystyrene/latex, polyethylene, polyamide, carboxyl modified teflon, nylon and nitrocellulose and metals and alloys such as gold, platinum and palladium. The solid support can be biological, non-biological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art. The surface of the solid substrate may contain reactive groups, such as carboxyl, amino, hydroxyl, thiol, or the like for the attachment of nucleic acids, proteins, etc. Surfaces on the solid substrate will sometimes, though not always, be composed of the same material as the substrate. Thus, the surface can be composed of any of a wide variety of materials, for example, polymers, plastics, resins, polysaccharides, silica or silica-based materials, carbon, metals, inorganic glasses, membranes, or any of the above-listed substrate materials.

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “a microorganism” includes more than one such microorganism. A reference to “a cell” includes more than one such cell, and so forth.

Like RT-RiboSyn, a FISH based method capable of measuring specific growth rate of a targeted microbial population must be capable of measuring the rate of ribosome synthesis by measuring the increase in new rRNA in individual cells over a period of time. Accumulation of precursor rRNA in cells treated with a protein synthesis inhibitor such as chloramphenicol provides an increasing pool of hybridization targets available for FISH. To this end, we used FISH, conducted with a fluorescently labeled probe that is designed to complement a unique site on the pre-16S rRNA of the Acinetobacter species, to measure the increase in mean whole cell fluorescence of actively growing cells treated with chloramphenicol. The linear increase in the mean whole cell fluorescence observed corresponds to a linearly increasing level of pre-16S rRNA in these cells. When this FISH based approach was applied to cells collected from stationary phase cultures of Acinetobacter species and treated with chloramphenicol, no appreciable increase in mean whole cell fluorescence was observed, indicating no accumulation of pre-16S rRNA. This clear distinction between growing and non-growing cells shows that FISH-RiboSyn has utility in applications where this distinction is necessary, such as antibiotic susceptibility testing.

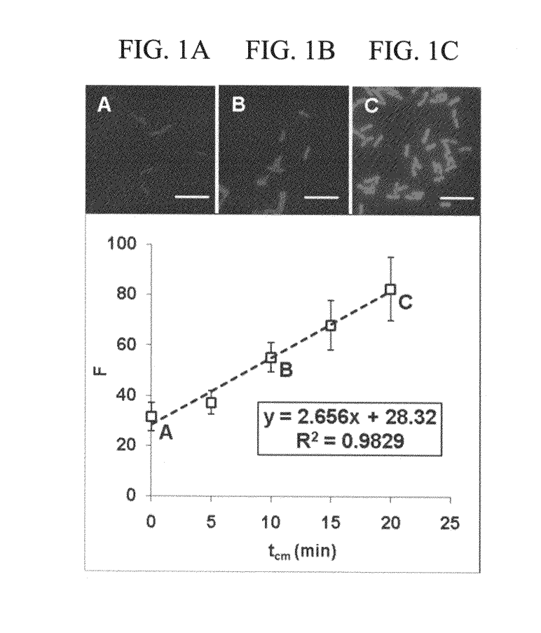

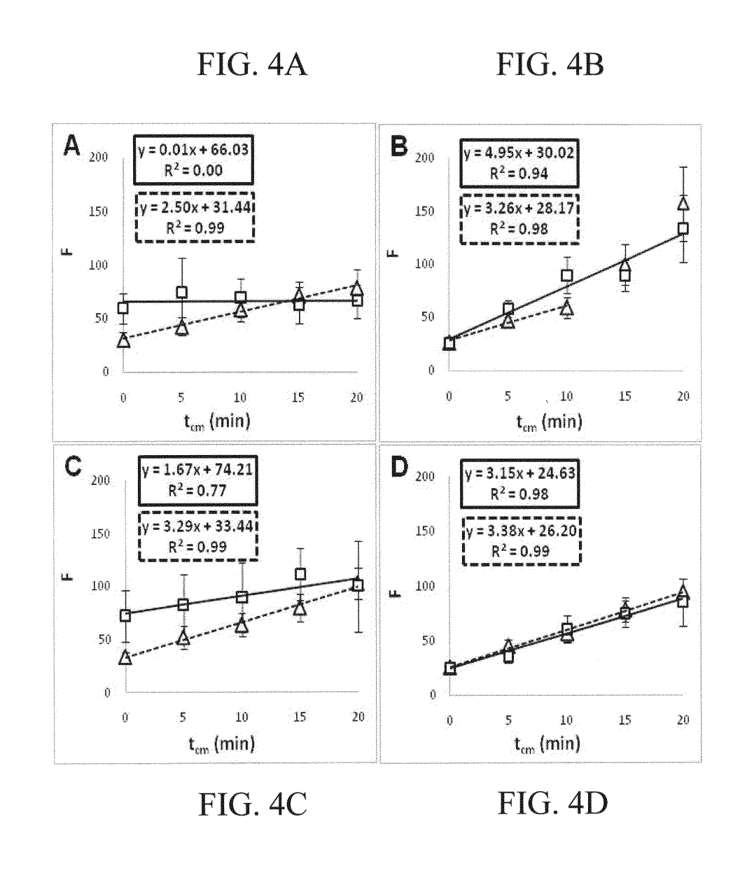

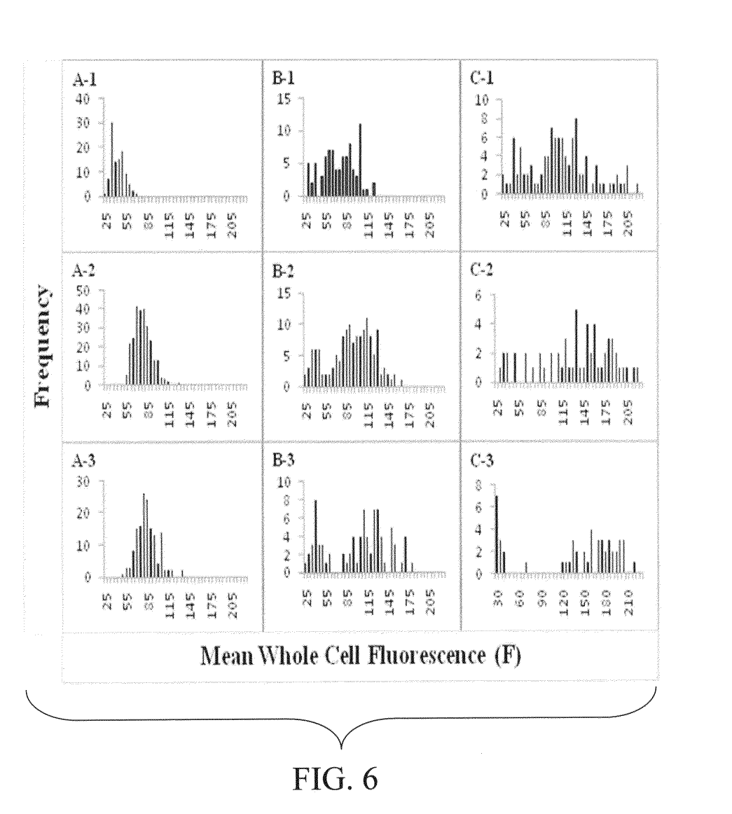

The use of the FISH-RiboSyn method for evaluating antibiotic susceptibility of A. baumannii strains revealed four distinct indicators, which may be used independently or in different combinations, to ascertain antibiotic susceptibility or resistance of clinical isolates (Table 4). The first indicator is the elevated initial mean whole-cell fluorescence (prior to chloramphenicol exposure) observed when a susceptible strain is exposed to either antibiotic for a short period (FIGS. 4A and 4C). In contrast, the resistant strain did not exhibit this effect for either antibiotic, which could be interpreted as an indication of resistance. The second indicator is the significant reduction in the rate of ribosome synthesis in the susceptible strain for both antibiotics, when cell growth ceased. In contrast, the resistant strain exhibited a much greater rate of ribosome synthesis, which may be an indication of resistance against both antibiotics. The third indicator is the increase in the average COV of the mean whole cell fluorescence from the five chloramphenicol treated samples used to calculate the rate of ribosome synthesis in the susceptible strain exposed to either antibiotic where growth has ceased. The fourth indicator is the development of a bimodal distribution of the mean whole cell fluorescence of individual cells of the susceptible strain exposed to levofloxacin. In contrast, the doxycycline treated cells of the susceptible culture did not exhibit a bimodal distribution of the mean whole cell fluorescence, but a broad normal distribution. The resistant strain did not exhibit a noticeable change in the distribution of the mean whole cell fluorescence of individual cells. It can be seen that at each level of analysis, a more accurate picture of the effect the antibiotic is having on a given organism type emerges. The bimodal distribution of F after 90 minutes of levofloxacin treatment (FIGS. 6B-1, 6B2, and 6B-3) may be a significant characteristic of the effects levofloxacin has on ribosome synthesis and can be used in addition to dF/dt_{cm} analysis as additional evidence that the cells are susceptible to the antibiotic. A similar effect was observed when A. lwoffii^{T} cells were treated with a lethal dose of ciprofloxacin (data not shown). As levofloxacin and ciprofloxacin are both fluoroquinolones, this bimodal distribution may be a unique characteristic of inhibition by this specific class of antibiotics. There is an obvious contrast between the observed effects of doxycycline (a tetracycline class antibiotic) and the fluoroquinolones on both the rate of ribosome synthesis as well as the distribution of pre-16S rRNA accumulation in individual cells (Table 4). In both cases an increase in mean whole-cell fluorescence was observed due to incubation of the susceptible strain with each antibiotic prior to treatment with chloramphenicol, as shown in FIGS. 4A-4D. However, in the case of levofloxacin, a large amount of variance is observed in the fluorescence measurements as a result of the observed bimodal distribution. These results may imply an additional effect levofloxacin has on the cell beyond its primary mechanism, which is to disrupt the function of topoisomerase IV, an important enzyme involved in DNA replication (Blondeau (2004)).

Through evaluation of pre-16SrRNA by FISH-RiboSyn it is evident that a clear distinction can be made between A. baumannii cells susceptible to doxycycline and levofloxacin and those which demonstrate resistance. Thus, the FISH-RiboSyn methods of the present invention is capable of determining antibiotic susceptibility in a number of relevant bacterial populations and warrants additional evaluation. However, evaluation of FISH-RiboSyn with cell wall synthesis inhibiting antibiotics may not be necessary as most of these compounds lead to cellular elongation, a trait which can readily be identified by cell staining and light microscopy. Since FISH-RiboSyn is designed to measure pre-16S rRNA molecules produced by a highly conserved anabolic pathway, the methods of the invention have potential for use in the evaluation of bacteria which acquired antibiotic resistance genes. In addition, the FISH-RiboSyn method is useful in identifying the antibiotic class and/or mechanism of inhibition of an uncharacterized compound, thereby making it a suitable tool for use in the drug discovery process.

The ability to properly assess the appropriate treatment of bacterial infections in a rapid and accurate fashion is a necessity for the success of clinical treatments, resulting in a decrease in patient mortality as well as addressing the growing concern of the emergence of antibiotic resistance due to improper administration of broad spectrum antibiotics. It is important to explore the power and accuracy associated with molecular biology based tools for the design of inexpensive, rapid assays which address sensitive clinical issues such as improper patient treatment and proliferation of antibiotic resistances to our limited pool of antibiotics.

### Materials and Methods for Examples 1-2

Culturing. Four Acinetobacter strains (Table 1) were maintained on nutrient agar (Difco) at 35° C. Prior to each experiment, a single colony was used to inoculate an overnight culture of nutrient broth (Difco). Experimental cultures were prepared by transferring 1 mL of culture to a flask containing 100 mL of nutrient broth. The relationship between optical density (OD at 600 nm) and cell concentration (CFU/mL) was determined by collecting samples between an OD of 0.1 and 0.6. The samples were serially diluted (10⁻⁴-10⁻⁷) in sterile nutrient broth and 100 μL of diluted sample was spread on nutrient agar plates in replicates of 5. After 24 hours of incubation at 35° C., colonies were counted on plates which contained between 20 and 200 colonies.

### Experiment I: Evaluation of Ribosome Synthesis in Acinetobacter spp.

Each bacterium was transferred from an overnight culture to a 500 mL culture flask containing 200 mL of sterile nutrient broth. Pure cultures were grown in a shaker incubator (200 rpm) at constant temperatures: A. calcoaceticus^{T} at 24° C., 30° C., and 35° C.; A. baumannii^{T} at 23° C., 30° C. and 35° C.; and A. lwoffii^{T} at 23° C., 30° C., and 35° C. Samples were also evaluated from a stationary phase culture for each species. Additionally, A. baumannii^{T} was grown in Smolders synthetic wastewater (per liter of water: 0.85 g CH₃COONa.3H₂O, 0.107 g NH₄Cl, 0.0755 g NaH₂PO₄.2H₂O, 0.09 g MgSO₄.7H₂O, 0.014 g CaCl₂. 2H₂O, 0.036 g KCl, 0.001 g yeast extract and 0.3 ml nutrient solution [per liter of water: 1.5 g FeCl₃.6H₂O, 0.15 g H₃BO₃, 0.03 g CuSO₄.5H₂O, 0.18 g KI, 0.12 g MnCl₂.4H₂O, 0.06 g Na₂MoO₄.2H₂O, 0.12 g ZnSO₄.7H₂O, 0.15 g CoCl₂.6H₂O, and 10 g EDTA]) at 30° C. and 200 rpm to facilitate slower growth (Smolders et al. (1994)). Optical density measurements were periodically taken and data was plotted in Excel (Microsoft) to generate a characteristic growth curve for each culture which was used to determine the actual value for μ attained for each culture by conventional, pure culture methods.

Sample collection. When it was observed that a culture was in mid-log growth phase (OD of approximately 0.4) 50 mL of the culture was transferred to a sterile flask and subjected to chloramphenicol at a final concentration of 200 mg/L. Samples (1 mL) were collected from the sub-culture every five minutes (including a sample at time zero) for a total of 20 minutes. Each sample was centrifuged at 10,000 G for 2 minutes, the supernatant was decanted, and the resulting cell pellet was resuspended in 1 mL of 4% PFA for 12-24 hours. The samples were centrifuged and supernatant decanted, as previously described, and resuspended in 2 mL of ethanol PBS (EtOH-PBS). The samples were stored at −20° C. until further analysis.

### Experiment II: Antibiotic Susceptibility Testing

To test the effects of the antibiotic doxycycline, doxycycline (Fisher Scientific) stock solution (1 mg/mL) was added to 100-mL of fresh, sterile nutrient broth for a final concentration of 8 μg/mL. A sample from an actively growing culture (10 mL) was added to the doxycycline/nutrient broth for a final volume of 110 mL. The aforementioned transfers were performed for both the resistant strain A. baumannii CBD1311 (OD=0.378) and a susceptible strain A. baumannii^{T} (OD=0.324). This procedure was repeated to test the effects of the antibiotic levofloxacin by adding levofloxacin (Sigma-Aldrich) stock solution (1 mg/mL) to 100-mL of fresh, sterile nutrient broth for a final levofloxacin concentration of 2 μg/mL. Transfers were performed for A. baumannii CBD1311 (OD=0.338) and A. baumannii^{T} (OD=0.365) and the dilutions were sufficient to ensure that the culture was below a McFarland standard of 0.5. Additionally, controls were implemented for each transfer where an additional 10 mL of the appropriate culture was combined with 100 mL of fresh sterile nutrient broth without an antibiotic. All cultures were incubated at 32° C. and shaken continuously (200 rpm). Samples were collected after 30, 90, and 150 minutes and processed for FISH-RiboSyn as described in Experiment I. Tests were performed by the CDB to determine the minimum inhibitory concentration (MIC) for both strains (Table 1).

Fluorescence in situ Hybridizations.

A fluorescently-labeled oligonucleotide hybridization probe, Acin1543, targeting the pre-16S rRNA for members of the genus Acinetobacter was synthesized (5′-GATTCTTACCAATCGTCAATCTTT-3′) (SEQ ID NO:1) and conjugated with the cyanine dye, Cy3, before purification with oligonucleotide probe purification cartridges (Oerther et al. (2000b)). Fluorescently labeled probes were diluted to 50 ng/μL with RNase-free water and stored at −20° C. in the dark. Fixed samples were applied to a sample well on a 10 well Heavy Teflon Coated microscope slide (Cel-Line Associates, New Field, N.J.) and air-dried. After dehydration with an increasing ethanol series (50, 80, 95% [vol/vol] ethanol, 1 min each), each sample well was covered with a mixture of 18 μL of hybridization buffer (20% [vol/vol] formamide, 0.9 M NaC1, 100 mM Tris HCl [pH 7.0], 0.1% SDS) (DelosReyes et al. (1997)) and 2 μL of the stock fluorescently labeled oligonucleotide probe. The hybridizations were conducted in a moisture chamber containing excess hybridization buffer (to prevent dehydration of buffer on sample wells) for 1.5 h, in the dark, at 46° C. The slides were washed for 30 min at 48° C. with 50 mL of pre-warmed washing buffer solution (215 mM NaCl, 20 mM Tris HCl [pH 7.0], 0.1% SDS, and 5 mM EDTA) (DelosReyes et al. (1997)). Fixed, hybridized cells were mounted with Type FF immersion oil (Cargille, Cedar Grove, N.J.) and a cover slip. Cells were stained with 4′, 6-diamidino-2-phenylindole (DAPI) at a concentration of 1 μg/mL for 1 minute and rinsed with DI water.

Image Capture. Whole cell fluorescence was visualized with an upright epiflourescence microscope (Leitz DiaPlan, Heerbrugg, Switzerland), and digital images were captured using a Spot-FLEX charge coupled device (CCD) camera (Diagnostic Instruments, Inc., Sterling Heights, Mich.). Images were collected using a 100× oil objective and constant exposure time (1.1 sec for experiment I; 3.0 sec for experiment 2) and gain of 2. The optimal image capture settings were determined by preliminary analysis of the fastest growing cells exposed to chloramphenicol for 20 minutes, which generated a maximum whole cell fluorescence of approximately 150 on an 8-bit scale.

Image Analysis. Images collected for experiment I were analyzed using the daime software package (Daims et al. (2006)). Five images were analyzed for each sample and manual thresholding was used to remove background (2D segmentation mode) and only whole cell fluorescence was retained for analysis. Each sample (series of five images) was processed with the same thresholding parameters and the “Measure objects” feature was used to calculate the number of discrete objects, the mean grey value of each object, and the standard deviation of the series.

For experiment II, five images from each sample were collected and analyzed using the Image-Pro Plus Version 6.2.0.424 software package (Media Cybernetics, Inc.). The “Count/size” feature of the software was used to group and isolate cells for fluorescence analysis. A size threshold (varied in size based on organism type and growth conditions) was used to ignore artifacts and isolate cell clusters no larger than 5 or 6 cells to prevent cell overlap or other phenomena which may interfere with proper quantification of mean whole-cell fluorescence. In most cases the “Automatic Bright Objects” counting method in the software was capable of isolating individual or small clusters of cells without inclusion of any background signal. Occasionally this feature would not return a rational result and a manual intensity range selection was performed. In each case all five images were analyzed and quantified by recording the number of objects within the thresholding range, the mean intensity of each object, the object area, and the standard deviation of the intensities. Intensity values are reported corresponding to an 8-bit range from 0-256.

Data Analysis. Optical density data, recorded during the mid-log growth phase of each culture, was used to calculate CFU/mL from the standard curve relating OD to CFU per mL. Values of CFU/mL were then plotted as a function of culture time in the form: 0.31×(t)=μ×log(CFU/mL)+b. The slope of the resultant plot was taken as μ (specific growth rate) and the standard error were determined by performing a linear regression analysis of the data in Excel. Mean whole cell florescence data was plotted as a function of chloramphenicol exposure time for each series of samples taken. Linear regression was then performed on the resultant plot to generate a slope (dF/dt_{cm}) (rate of ribosome synthesis). For the data collected from experiment I, a value of dF/dt_{cm} was calculated at each unique growth condition and plotted as a function of the specific growth rate as determined by optical density analysis. For each value of dF/dt_{cm}, the average coefficient of variance (COV) was calculated for all data used in the linear regression.

For data collected in experiment II, linear regression was used to determine dF/d_{cm} as in experiment I. The value of dF/dt_{cm} for each antibiotic at each time of exposure was plotted alongside the appropriate control to demonstrate the effects that antibiotic treatment have on ribosome synthesis (dF/dt_{cm}). Histograms were generated from data collected by image analysis of levofloxacin treated cells. Object counts were separated by mean whole cell fluorescence and organized in bins with a size of 5 intensity units. The bin range was taken from 25-225 where any value which falls outside of this range is included in the closest relevant bin.

Following are examples that illustrate procedures for practicing the invention. These examples should not be construed as limiting. All percentages are by weight and all solvent mixture proportions are by volume unless otherwise noted.

### EXAMPLE 1

### EVALUATION OF RIBOSOME SYNTHESIS IN ACINETOBACTER SPP

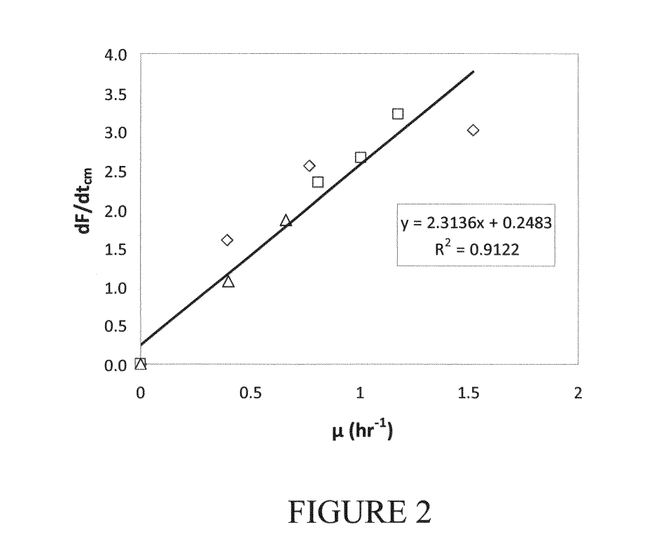

For each actively growing Acinetobacter culture, a linear increase in mean whole cell fluorescence intensity over the duration of chloramphenicol exposure was observed as shown in FIGS. 1A-1C. The initial, weak fluorescent signal (FIG. 1A) is due to the low steady state concentration of pre16S-rRNA, which is typical of healthy growing cells (Licht et al. (1999)). As chloramphenicol begins to inhibit secondary rRNA processing, a buildup of cellular pre16S-rRNA occurs and is directly observed as an increase in mean whole cell fluorescence (FIGS. 1B and 1C) (Oerther et al. (2000a)). A strong linear correlation between F and t_{cm} is observed, however the variance increases with higher mean whole cell fluorescence. This increase in variance is the result of the greater difference between the fluorescence of the pixels near the center of the object (cell) and at the edge of the object for brighter objects. An outline of the growth data acquired by optical density analysis is presented alongside the results of image analysis in Table 2. From this it can be shown that dF/dt_{cm} increases with increased μ for cells in constant growth. A good linear relationship between F and t_{cm} is observed for actively growing cultures with high values of R² ranging from 0.933 to 0.997. The dF/dt_{cm} is near zero in stationary phase cultures and ranges from 1.07 to 3.22 for actively growing cultures. The COV for each dF/dt_{cm} was between 9.8% and 15.9% for actively growing cells, which is consistent with typical FISH image analysis. However, the stationary phase cultures had COV for each dF/dt_{cm} that were 5.3%, 8.5%, and 13.2%, which is consistent with low mean whole cell fluorescence and dF/dt_{cm} typical of non-growing cultures. When all three species are collectively analyzed by comparing dF/dt _{cm} to the respective g, a strong linear relationship is observed (FIG. 2). This collective analysis of all FISH image data for all Acinetobacter species is possible when the FISH and image collection protocols are identical. This strong linear relationship suggests a tight coupling of the rate of ribosome synthesis within the Acinetobacter species.

Our results (FIG. 2) also suggest that as μ approaches μₘₐₓ, there appears to be a maximum rate of ribosome synthesis (dF/dt_{cm}=3.22) for Acinetobacter

$$\frac{d }{d μ} ( \frac{d F }{d t} ) = 0.$$

The highest reported μₘₐₓ is 1.28 hr⁻¹ for A. calcoaceticus (Dupreez and Toerien (1978)), which is very close to our results (1.18 hr⁻¹). However, A. baumannii^{T} has a higher maximum μ of 1.52 hr⁻¹, but does not exhibit the same trend as the other species. This may indicate that A. baumannii^{T} can grow at a faster μ than the other Acinetobacter species despite the limitation of a maximum rate of ribosome synthesis. Studies utilizing cells collected from a chemostat may be helpful in clarifying this observation. The relationship between dF/dt_{cm} and μ for Acinetobacter may be indicative of a similar relationship for other genera.

Distinct morphological differences amongst the Acinetobacter species evaluated were observed during FISH analysis that include diplobacillus (A. calcoaceticus^{T}), diplococcus (A. lwoffii^{T}), and growth dependent variability (A. baumannii^{T}). Acinetobacter baumannii^{T} has been reported to vary in cell morphology depending on growth condition, stage of cell growth, and/or the presence of antibiotics (Bayuga et al. (2002)). Despite the range of morphologies, the data uniformity suggests that the method is insensitive to cell morphology making it potentially useful for a wide range of bacteria morphotypes.

### EXAMPLE 2

### ANTIBIOTIC SUSCEPTIBILITY TESTING

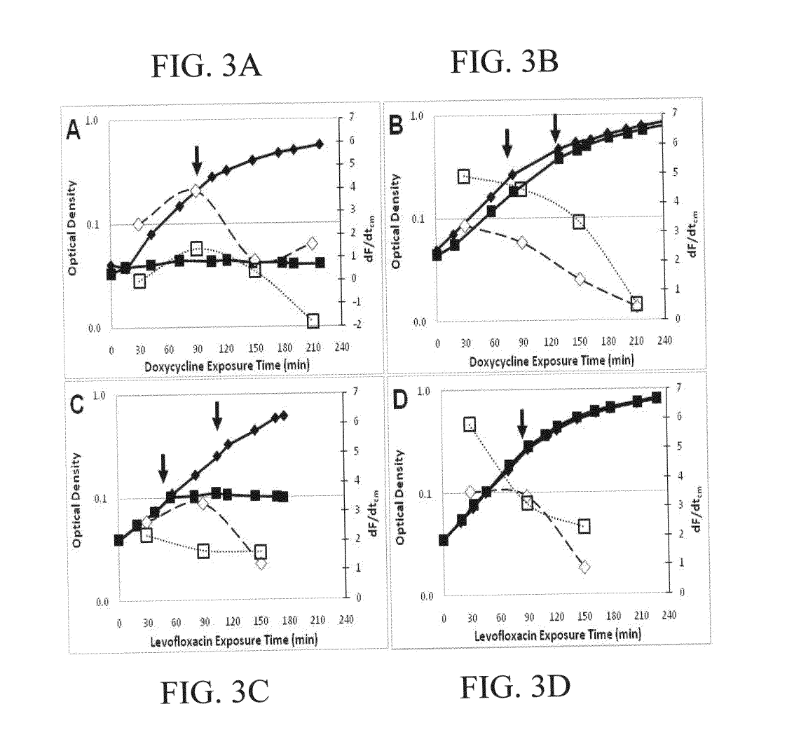

The FISH-RiboSyn method was used to evaluate antibiotic susceptibility and resistance in A. baumannii strains for two antibiotics: doxycycline, a bactericidal protein synthesis inhibitor; and levofloxacin, a bacteriostatic DNA replication disruptor. For each antibiotic, a susceptible strain, A. baumannii^{T}, and resistant strain, A. baumannii CBD1311, was treated with the antibiotic and dF/dt_{cm} was determined at various antibiotic exposure times and compared to its respective control (i.e., untreated culture). For susceptible cultures (FIGS. 3A and 3C), the OD data clearly portrays the antibiotic effect of the treated cells while untreated cells remain unaffected. The doxycycline immediately suppressed growth of the susceptible strain, while treatment with levofloxacin exhibited a 60 minute delay before cessation of growth. In contrast, the resistant and control cultures have nearly identical growth profiles, although there does appear to be a very slight suppression associated with doxycycline treatment (FIG. 3B).

For doxycycline treated cultures, there is a distinct difference between dF/dt_{cm} for A. baumannii^{T} (2.5) in comparison to the control (0.01) after only 30 minutes of antibiotic exposure (FIG. 3A). After 90 minutes however, there is a slight increase in dF/dt_{cm} for the treated cells (1.38), but is still very low in comparison to the control (3.93) providing distinct evidence of antibiotic impact. Beyond log growth (as marked by the arrow), dF/dt_{cm} is drastically lower for all cultures observed and is due to a reduction in the rate of ribosome synthesis as cells prepare for entry into stationary phase.

For analysis of antibiotic susceptibility, we did not consider data collected from cultures beyond the log growth phase. In the case of A. baumannii CBD1311, after 30 minutes of exposure to doxycycline, dF/dt_{cm} of the treated cells (4.95) was much higher compared to the control (3.26) as shown in FIG. 3B. After both cultures exited log growth phase, dF/dt_{cm} decreased. Little impact in dF/dt_{cm} was observed after 30 minutes of exposure to levofloxacin in susceptible cells (2.23) when compared to the control (2.68) as is consistent with the OD data (FIG. 3C). After 60 minutes of exposure to levofloxacin, however; A. baumannii^{T} demonstrated a dramatic impact in dF/dt_{cm} (1.67) compared to the control (3.29). Evaluation of A. baumannii CBD 1311 treated with levofloxacin shows a peculiarity in the response of individual cells. Although growth data suggests no antibiotic impact, dF/dt_{cm} for the treated cells is significantly higher (5.84) in comparison to the control (3.55) as shown in FIG. 3D. When A. baumannii CBD1311 is treated with either antibiotic for a short period of time (i.e., 30 min), the rate of ribosome synthesis appears to be much higher relative to the observed μ based on the OD data from the control and antibiotic treated cultures. This may be a unique indicator of doxycycline and levofloxacin resistance. After 90 minutes of levofloxacin treatment, dF/dt_{cm} is very similar for both the treated cells and the control, which suggests similar growth.

Uniformity, as defined by the average COV of the mean whole cell fluorescence data used to determine dF/dt_{cm}, is consistent with a range of 5.3% to 15.9% for all cultures not treated with an antibiotic (Table 2). Both doxycycline and levofloxacin impacted cells of A. baumannii^{T} show an increase in the average COV compared to the control (Table 3), which may be indicative of inhibited growth. In contrast, A. baumannii CBD1311 exhibited similar average COV for cells treated with either antibiotic compared to the control.

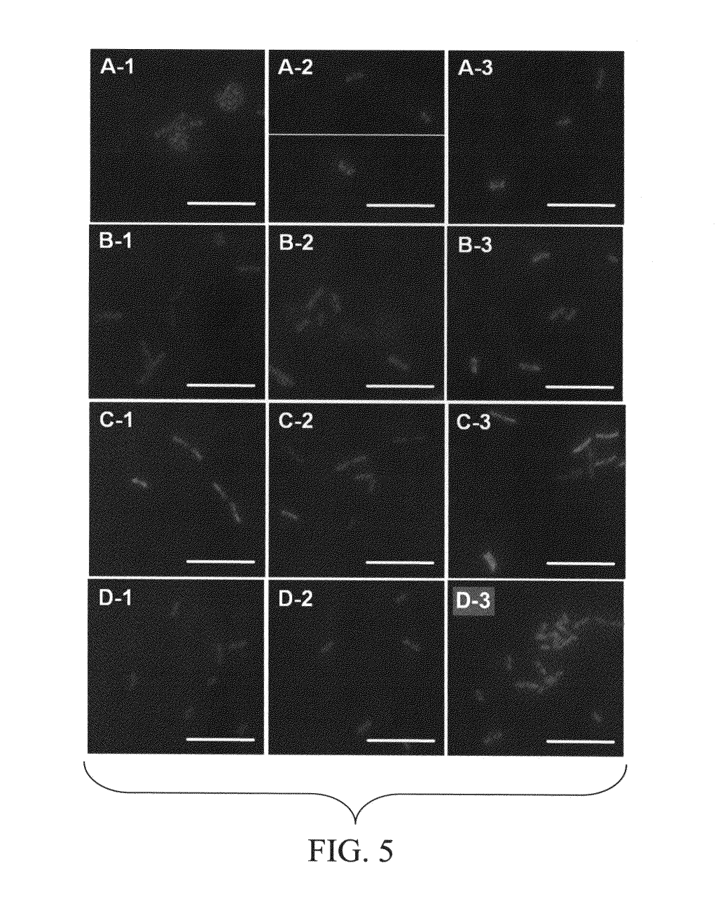

FIGS. 4A-4D provides a clear presentation of the effects each antibiotic has on the rate of ribosome synthesis with corresponding representative FISH images provided in FIGS. 5A-1 through 5D-3. A. baumannii^{T} shows strong inhibition of ribosome synthesis with a near zero dF/dt_{cm} after only 30 minutes of exposure to doxycycline (FIGS. 4A, 5A-1, 5A-2, and 5A-3), while the dF/dt_{cm} of the control was 2.50 (FIGS. 4A, 5B-1, 5B-2, and 5B-3), which is consistent with normal growing cells (FIGS. 1A-1C). Additionally, the initial mean whole cell fluorescence of the treated culture (59.6) was elevated compared to the control (30.7). In contrast, A. baumannii CBD1311 has a greater dF/dt_{cm} of 4.95 compared to the control (3.26) as shown in FIG. 4B, although the initial mean whole cell fluorescence was approximately 26 for both cultures. Data collected at chloramphenicol exposure times of 15 and 20 minutes for the control culture presented in FIG. 4B were omitted from the linear regression used to calculate dF/dt_{cm} due to a high level of artifacts in the images which generated a higher value of F than is representative of this particular culture condition. Subsequent FISH analysis of both doxycycline treated and control samples revealed a comparable trend to the trends presented in FIG. 4B. Although the subsequent analysis provided confidence in the omission of these data points, the data collected from that analysis could not be included due to aging of the cells and reduction in fluorescent lamp intensity which ultimately reduces the mean whole cell fluorescence of all samples. For levofloxacin, A. baumannii^{T} shows clear evidence of antibiotic impact on ribosome synthesis after 90 minutes of exposure (FIG. 4C) with over a 50% reduction in dF/dt_{cm} (1.67; FIGS. 5C-1, 5C-2, and 5C-3) compared to the control (3.29; FIGS. 5D-1, 5D-2, and 5D-3). Additionally, levofloxacin treated cells showed a highly elevated initial mean whole cell fluorescence of 71 compared to the control (31). Although the data does not suggest complete inhibition of ribosome synthesis, there are clear signs of antibiotic impact. Levofloxacin treated A. baumannii CBD1311 shows no observable impact on the rate of ribosome synthesis and no difference in the initial mean whole cell fluorescence (FIG. 4D).

FIGS. 5C-1, 5C-2, and 5C-3 reveal an additional effect that levofloxacin has on the level of pre-16S rRNA of individual cells, where some cells exhibit a much greater mean whole cell fluorescence intensity than others. A more thorough analysis of this observation was possible by examination of histograms of the mean fluorescence intensity of individual cells (FIGS. 6A-1 through 6C-3). After 30 minutes of treatment with levofloxacin, A. baumannii^{T} shows a typical increase in F over the duration of chloramphenicol treatment with a normal distribution, which is typical of growing cells (FIGS. 1A-1C). In contrast, after 90 minutes of levofloxacin treatment the cells are not growing (FIG. 3C) and begin to demonstrate a broader distribution of F prior to chloramphenicol treatment (FIG. 6B-1), which culminates after 20 minutes of chloramphenicol treatment into a bimodal distribution with the bimodal means of approximately 40 and 130 (FIG. 6B-3). After 150 minutes of levofloxacin exposure a bimodal distribution is evident after 20 minutes of chloramphenicol exposure (FIG. 6C-3) and the distribution of F is substantially broadened in comparison to the 90 minute exposure data. Within the culture treated with levofloxacin for extended periods, a subpopulation is accumulating pre-16S rRNA prior to chloramphenicol treatment (FIGS. 6A-1, 6B-1, and 6C-1). When cultures are incubated with levofloxacin for 90 and 150 minutes and subsequently treated with chloramphenicol (FIGS. 6B-1, 6B-2, and 6B-3 and FIGS. 6C-1, 6C-2, and 6C-3), a greater shift in the subpopulation with the high mean whole cell fluorescence is observed compared to culture incubated with levofloxacin for 30 minutes (FIGS. 6A-1, 6A-2, and 6A-3). This suggests that the levofloxacin treatment alone is causing over expression of the rrn operons and possibly inhibiting pre-16S rRNA maturation. This development of a bimodal distribution also explains the persistent and elevated dF/dt_{cm} (FIG. 3C) and high mean COV (Table 3) despite cessation of growth; however, there is a clear and distinct difference in dF/dt_{cm} when compared to actively growing cells.

TABLE 1
Acinetobacter strains used in this study and information
regarding their antibiotic susceptibility.

| | | doxycycline | | levofloxacin | |
|---|---|---|---|---|---|
| | | MIC | Dose | MIC | Dose |
| | ATCC # | (μg/mL) | (μg/mL) | (μg/mL) | (μg/mL) |
| Acinetobacter | 23055 | N/A | N/A | N/A | N/A |
| calcoaceticus^{T} | | | | | |
| Acinetobacter | 15309 | N/A | N/A | N/A | N/A |
| lwoffii^{T} | | | | | |
| Acinetobacter | 19606 | ≦0.125 | 8 | 0.25 | 2 |
| baumannii^{T} | | | | | |
| Acinetobacter | N/A | 16 | 8 | 8 | 2 |
| baumannii CBD1311 | | | | | |

TABLE 2
Specific growth rate as determined by OD measurements for
each Acinetobacter strain at various culturing temperatures
(including stationary phase) as well as the corresponding
slopes (dF/dt_{cm}) and coefficients of determination (R²)
for each linear regression performed on data collected
from image analysis. The OD represents the OD of the
culture when a sample was removed and subjected to
chloramphenicol.

| | | OD for | | | | |
|---|---|---|---|---|---|---|
| | Temp | FISH- | μ | | | Average |
| | (° C.) | RiboSyn | (hr⁻¹) | dF/dt_{cm} | R² | COV |
| Acinetobacter | 30 [symbol] | 0.302 | 0.392 | 1.60 | 0.942 | 12.3% |
| baumannii^{T} | 23 | 0.439 | 0.772 | 2.55 | 0.997 | 15.9% |
| | 35 | 0.532 | 1.52 | 3.02 | 0.995 | 14.8% |
| | Stationary | 1.07 | 0 | −0.036 | 0.5 | 8.5% |
| Acinetobacter | 24 | 0.477 | 0.810 | 2.34 | 0.993 | 13.4% |
| calcoaceticus^{T} | 30 | 0.465 | 1.18 | 3.22 | 0.933 | 13.7% |
| | 35 | 0.373 | 1.01 | 2.66 | 0.983 | 14.2% |
| | Stationary | 0.72 | 0 | −0.004 | 0.125 | 13.2% |
| Acinetobacter | 23 | 0.364 | 0.398 | 1.07 | 0.985 | 11.8% |
| lwoffii^{T} | 30 | 0.354 | 0.660 | 1.87 | 0.982 | 9.8% |
| | Stationary | 0.67 | 0 | 0.046 | 0.568 | 5.3% |

[symbol]

culture was grown in Smolder's media.

TABLE 3
Values of the average COV of the mean whole cell fluorescence
used to generate individual values of dF/dt_{cm} for tests
performed on both antibiotics.

| Exposure time | A. baumannii^{T} | | A. baumannii CBD1311 | |
|---|---|---|---|---|
| (min) | control | doxycycline | control | doxycycline |
| 30 | 19.8% | 28.8% | 17.1% | 16.5% |
| 90 | 18.4% | 22.8% | 16.5% | 15.5% |
| 150 | 16.3% | 24.8% | 13.0% | 14.6% |
| | control | levofloxacin | control | levofloxacin |
| 30 | 16.2% | 17.2% | 15.5% | 13.9% |
| 90 | 15.9% | 34.1% | 12.5% | 17.3% |
| 150 | 18.0% | 43.8% | 16.7% | 15.2% |

TABLE 4
Summary of antibiotic susceptibility/resistance indicators as
measured by FISH-RiboSyn for the two strains treated with
doxycycline and levofloxacin.

| | indicator | A. baumannii^{T} | A. baumannii CBD1311 |
|---|---|---|---|
| Doxycycline | Mean Fᵢₙᵢₜᵢₐₗ | ↑ | normal |
| | dFdt_{cm} | ↓ | ↑ |
| | COV | ↑ | normal |
| | F distribution | wide normal | normal |
| Levofloxacin | Mean Fᵢₙᵢₜᵢₐₗ | ↑ | normal |
| | dFdt_{cm} | ↓ | ↑ |
| | COV | ↑↑ | normal |
| | F distribution | bimodal | normal |

### Materials and Methods for Example 3-6

### Cell Culture and Sample Collection

Acinetobacter lwoffii (ATCC 15309) was cultured on nutrient agar plates inoculated from a frozen glycerol/bead stock. After incubation for 24 hours at 32° C., a single colony was removed from the plate with a sterile loop and streaked on a second nutrient agar plate. The sterile loop was then submerged in a test tube containing 10 mL of nutrient agar which was incubated for 12 hours and 1 mL of grown culture was added to 1 mL of 4% paraformaldehyde (PFA) and incubated for 1 hour. The sample was then centrifuged at 10,000 G for 5 minutes, the supernatant decanted, and the cell pellet resuspended in ethanol PBS. Fluorescence in situ hybridization (FISH) with a genus specific probe was performed on this sample to verify culture purity before proceeding with the experiment. After culture purity was verified, the plated culture was incubated for 24 hours and a single colony was picked with a sterile loop and transferred to a 10 mL test tube of nutrient broth and incubated for 14 hours. The primary culture for this experiment was inoculated by transferring 1 mL from the test tube to a 100 mL nutrient broth flask. The optical density (OD at wavelength of 600 nm) of the culture was monitored over time until OD=0.449. At this time 10 mL of culture was transferred to four separate flasks containing 90 mL of nutrient broth and an appropriate amount of antibiotic. Flask 1 was treated with 400 μL of ampicillin solution (500 μg/mL) for an effective concentration of 2 μg/mL which has been shown to be lethal to A. lwoffii (Seifert et al. (1993)). Flask 2 was treated with 70 μL of ciprofloxacin solution (357 μg/mL) for an effective concentration of 0.25 μg/mL which has been shown to be lethal to A. lwoffii (Seifert et al. (1993)). Flask 2 was treated with 1 mL of doxycycline solution (50 mg/mL) for an effective concentration of 500 μg/mL which has been shown to be a lethal dose for Acinetobacter spp. (Wisplinghoff et al. (2000)). Flask 4 was untreated as a control culture. At the same time, 9 mL of the primary culture was added to a 50 mL conical containing 1 mL of 1 mg/mL chloramphenicol for a final concentration of 100 μg/mL. Samples of 1 mL were taken from the chloramphenicol treated cell culture at 0, 5, 10, 15, and 20 minutes and incubated in 1 mL of 4% PFA for at least one hour. These samples were then centrifuged at 10,000 G for 5 minutes, the supernatant decanted and cell pellets resuspended in ethanol PBS and stored at −20° C. until evaluated by FISH.

It is important to note that aforementioned lethal doses of antibiotic are generally suitable for cultures that have a turbidity equal to or less than a McFarland standard solution of 0.5 which generally represents a cell concentration of approximately 1.5×10⁸ CFU/mL (Andrews (2001)). A dilution series of a growing culture of A. lwoffii was previously used to determine the relationship between OD (at 600 nm) and CFU/mL by plating diluted samples on nutrient agar plates at various values of OD and performing plate counts. It was determined that a cell concentration of 1.5×10⁸ CFU/mL occurs at OD=0.217 for A. lwoffii in nutrient broth. Since samples in this experiment were collected at OD=0.45 and then diluted more than tenfold (15 mL of culture added to 90 mL of fresh nutrient broth media) we are confident that the concentration of antibiotic used in each case exceeds the minimum inhibitory concentration (MIC) and can be considered lethal.

Treated flasks were allowed to incubate for 30 minutes and which time 9 mL of the primary culture was added to a 50 mL conical containing 1 mL of 1 mg/mL chloramphenicol for a final concentration of 100 μg/mL. Samples of 1 mL were taken from the chloramphenicol treated cell culture at 0, 5, 10, 15, and 20 minutes and incubated in 1 mL of 4% PFA for at least one hour. These samples were then centrifuged at 10,000 G for 5 minutes, the supernatant decanted and cell pellets resuspended in ethanol PBS and stored at −20° C. until evaluated by FISH. This was repeated for each flask at 90, 150, and 210 minutes of incubation with the antibiotic.

### Image Analysis

All images were analyzed using the Image-Pro Plus (v6.2.0.424) software package. Only single cells or diplococci were included in analysis. Images were thresholded using the “Automatic Bright Objects” option in the measurement console. Occasionally this algorithm excluded dim objects if bright objects were present. In these cases the manual thresholding was used to find the optimal thresholding range. Object sizes were within the range from 30-200 pixels² except for the analysis of ampicillin treated cells which enlarged over the course of antibiotic exposure. The highest size threshold used for these cells was 5,000 pixels². Thresholded and size filtered objects were used to determine the mean whole-cell fluorescence and this data was used to evaluate the effect of each antibiotic.

Following are examples that illustrate procedures for practicing the invention. These examples should not be construed as limiting. All percentages are by weight and all solvent mixture proportions are by volume unless otherwise noted.

### EXAMPLE 3

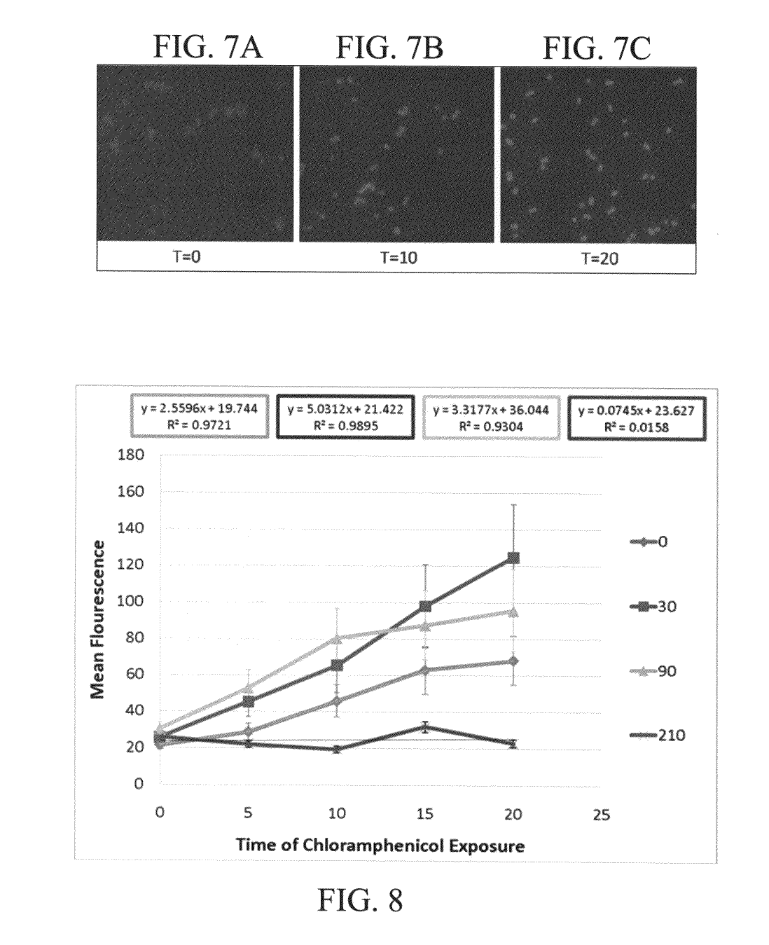

The increase in mean whole-cell fluorescence intensity as a function of chloramphenicol exposure for untreated cells is presented in FIGS. 7A-7C and a graphical representation is provided in FIG. 8.

As previously observed, a strong linear relationship between mean whole-cell fluorescence and exposure time to chloramphenicol is presented. This is due to build-up of precursor rRNA in the chloramphenicol inhibited cells and represents the rate at which the cell is synthesizing new ribosomes.

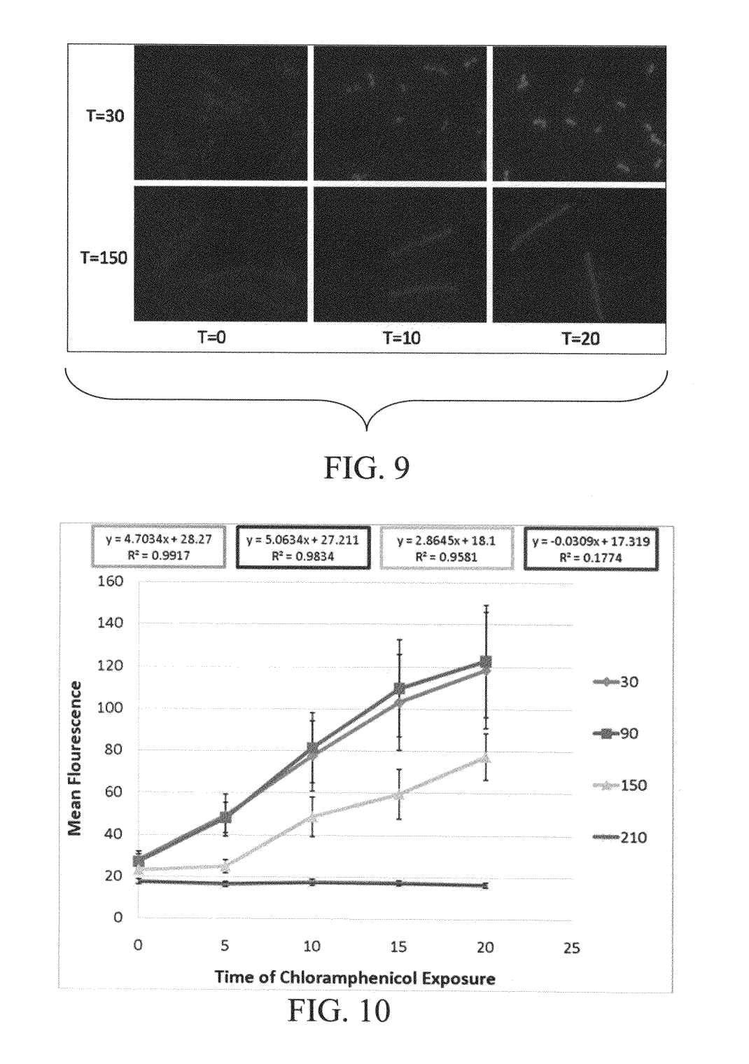

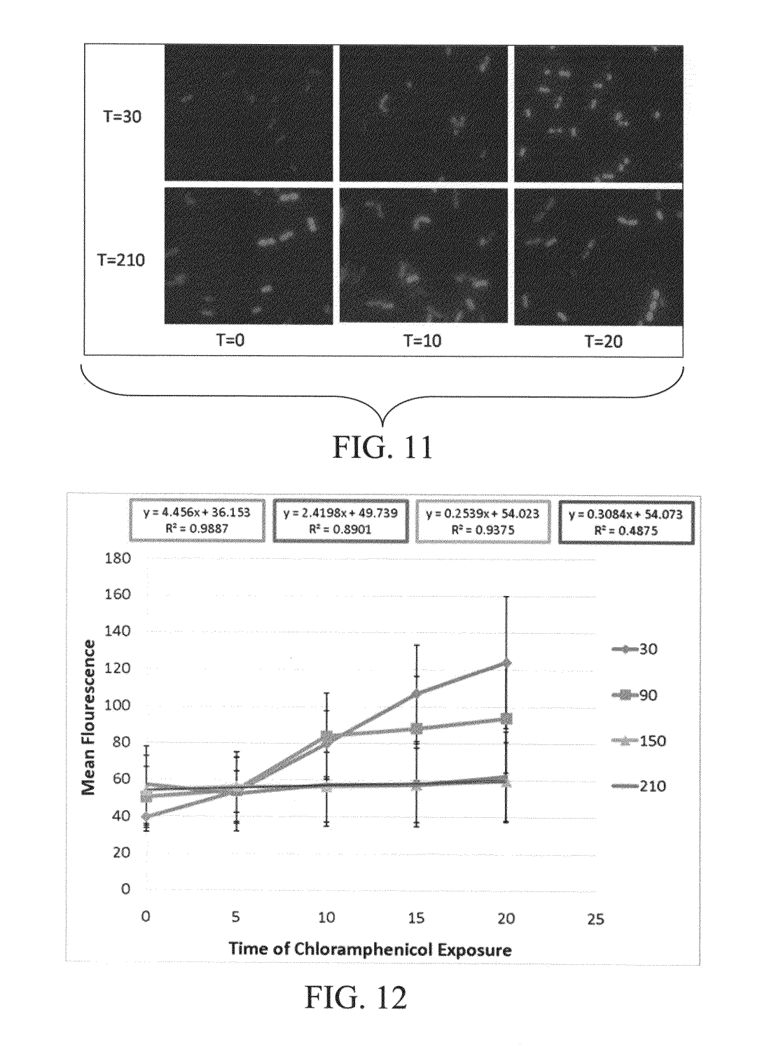

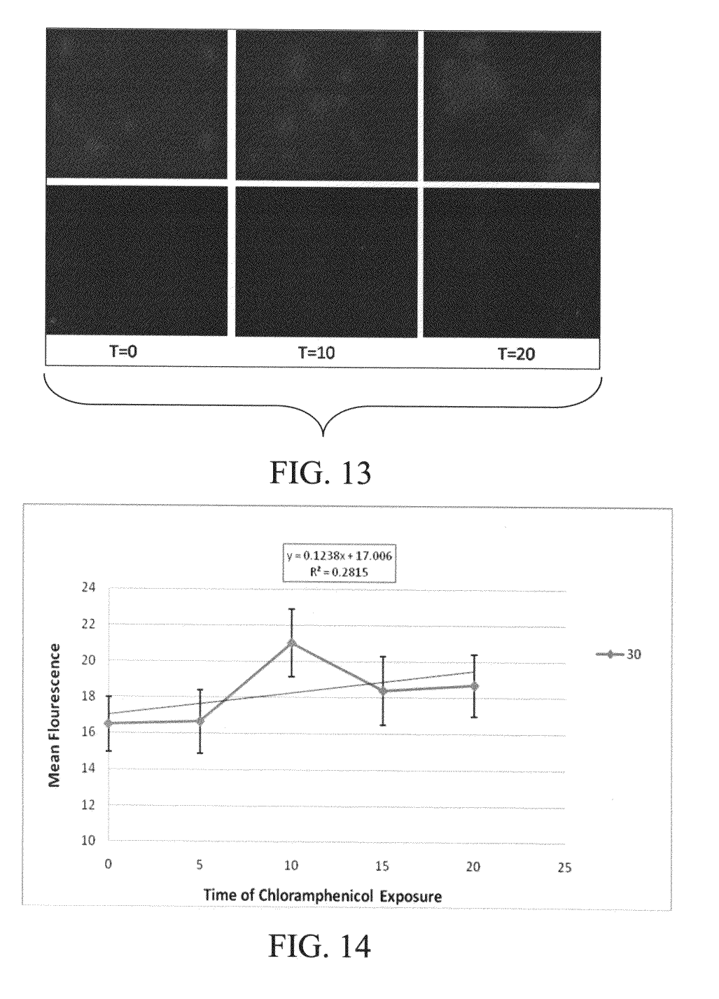

FISH images were collected and analyzed in a similar fashion for A. lwoffii cells exposed to three antibiotics that represent three distinct classes of antibiotics (Table 5). The relationship for cells treated with Ampicillin (FIGS. 9 and 10), Ciprofloxacin (FIGS. 11 and 12) and Doxycycline (FIGS. 13 and 14) are described herein.

TABLE 5
Cellular targets of antibiotics.

| Target | Antibiotics |
|---|---|
| Cell wall synthesis | Polypeptides |
| | β-Lactams |
| | Glycopeptides |
| Cell membrane structure and function | Polypeptides |
| Folic acid synthesis | Trimethoprim |
| | Sulfonamides |
| DNA structure and function | Quinolones |
| | Fluoroquinolones |
| | Nitrofurantoin |
| | Nitrimidazole |
| Transcription | Rifampin |
| Protein synthesis | Aminoglycosides |
| | Clindamycin |
| | Lincosamide |
| | Macrolides |
| | Tetracyclines |

### EXAMPLE 4

### AMPICILLIN TREATED CELLS

Ampicillin is a β-Lactam antibiotic which disrupts cell wall synthesis (Table 5). As expected, long exposures to ampicillin resulted in elongated cells (T=150 min). Ribosome synthesis is impacted by the ampicillin after exposure for 150 and 210 min (FIG. 10).

### EXAMPLE 5

### CI*PROFLOXACIN TREATED CELLS

Ciprofloxacin is a fluorquinolone antibiotic which disrupts DNA structure and function (Table 5). Unexpectedly, ciprofloxacin treated cells of A. lwoffii produced a bimodal distribution of cells where a subpopulation of cells have high initial cell fluorescence (T=210 min), while other cells have low fluorescence. This subpopulation of high initial cell fluorescence increases in size with longer exposure to ciprofloxacin. The dF/dt decreases with longer exposures to ciprofloxacin. Cells appear to not be synthesizing ribosomes after 150 min of exposure to ciprofloxacin.

### EXAMPLE 6

### DOXYCYCLINE TREATED CELLS

Doxycycline is a tetracycline antibiotic which disrupts protein synthesis (Table 5). Cells of A. lwoffii are impacted immediately by doxycycline with cessation of ribosome synthesis detected after 30 minutes of exposure. Image analysis for cells treated with doxycycline for more than 30 minutes of exposure were not included, because cells could not be distinguished from the background. DAPI stained cells are visible (FIG. 13), but precursor 16S rRNA levels are not detectable.

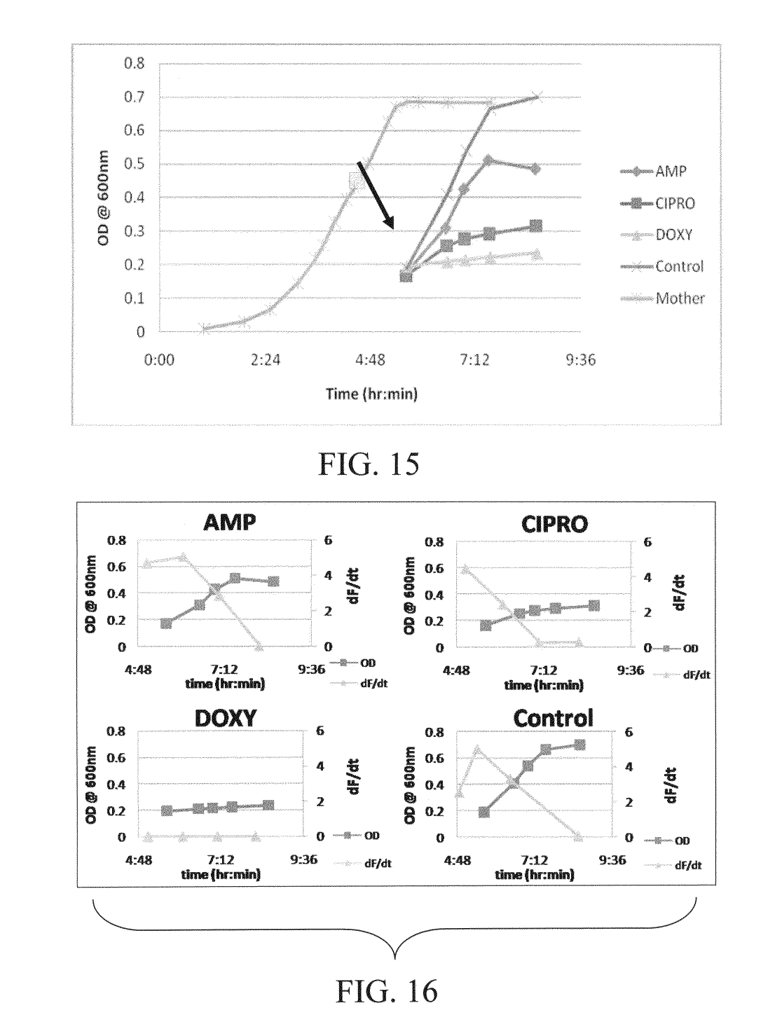

The optical densities of the mother culture and both control and experimental batch cultures are provided in FIG. 15. As expected, the control batch culture reached a similar maximum OD as the mother culture. The optical densities of the three experimental batch reactors were impacted by the antibiotics with ampicillin producing a mild impact compared to ciprofloxacin and deoxycycline.

For comparison, the dF/dt was plotted with the optical density for the control and experimental batch reactors and is shown in FIG. 16. For ciprofloxacin and deoxycycline, the impact of these antibiotics on ribosome synthesis is evident. Ampicillin treated cells have a similar dF/dt profile as the control, but there are clear differences. Short exposure to ampicillin generates a greater dF/dt than the control cells, which suggests that these cells are synthesizing ribosomes at a faster rate.

A comparison of dF/dt for each experimental batch reactor to the control batch reactor is shown in Table 6.

TABLE 6
Values of dF/dt measured for the control and experimental batch
cultures at increasing times of antibiotic exposure (top) and
the ratio of each rate compared to the control at that time.

| | | Antibiotic Exposure (min) | | | | |
|---|---|---|---|---|---|---|
| | | 0 | 30 | 90 | 150 | 210 |
| dF/dt | Control | 2.56 | 5.03 | 3.32 | NA | 0.07 |
| | Ampicillin | NA | 4.7 | 5.06 | 2.86 | 0 |
| | Ciprofloxacin | NA | 4.46 | 2.42 | 0.254 | 0.308 |
| | Doxycycline | NA | 0.1238 | 0 | 0 | 0 |
| | AMP/CT | NA | 93% | 152% | NA | 0% |
| | CIP/CT | NA | 89% | 73% | NA | 440% |
| | DOXY/CT | NA | 2% | 0% | NA | 0% |

Overall these Results Show the Potential of Using FISH-RiboSyn for Antibiotic Susceptibility Testing (AST) of Pathogenic Bacteria.

All patents, patent applications, provisional applications, and publications referred to or cited herein are incorporated by reference in their entirety, including all figures and tables, to the extent they are not inconsistent with the explicit teachings of this specification. It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,458,066
U.S. Published Application No. US-2008-0009011
AdvanDx, (2009) “AdvanDx Receives FDA 510(k) Clearance for EK/P. aeruginosa PNA FISH®”
Agrawal, S. (Ed.) (1993) “Protocols for Oligonucleotides and Analogues; Synthesis and Properties,” Methods in Molecular Biology Series; Volume 20; 1^{st} Edition, Humana.
Amgen, (2009) “New survey highlights growing concern about risk of infection in cancer patients”
Andrews, J. M. (2001) “Determination of minimum inhibitory concentrations” Journal of Antimicrobial Chemotherapy 48:5-16.
Ausubel et al. (1989), Current Protocols in Molecular Biology, Green Publishing Associates and Wiley Interscience, N.Y.
Bayuga, S., C. Zeana, J. Sahni, P. Della-Latta, W. El-Sadr, and E. Larson (2002) “Prevalence and antimicrobial patterns of Acinetobacter baumannii on hands and nares of hospital personnel and patients: The iceberg phenomenon again” Heart & Lung 31:382-390.
Beaucage et al., Tetrahedron Lett. 22:1859-62 (1981)
Beltz et al. (1983) Methods of Enzymology, R. Wu, L. Grossman and K. Moldave [eds.] Academic Press, New York 100:266-285.
Blondeau, J. M. (2004) “Fluoroquinolones: Mechanism of action, classification, and development of resistance” Survey of Ophthalmology 49:S73-S78.
Brown et al., Meth. Enzymol. 68:109-51 (1979)
Cangelosi, G. A. and W. H. Brabant (1997) “Depletion of pre-16S rRNA in starved Escherichia coli cells” Journal of Bacteriology 179:4457-4463.
CDC, (1999) “Control of infectious diseases” MMWR Morb Mortal Wkly Rep 48:621-9.
Cutter, M. R. and P. G. Stroot (2008) “Determination of specific growth rate by measurement of specific rate of ribosome synthesis in growing and nongrowing cultures of Acinetobacter calcoaceticus” Applied and Environmental Microbiology 74:901-903.
Daims, H., S. Lucker, and M. Wagner (2006) “Daime, a novel image analysis program for microbial ecology and biofilm research” Environmental Microbiology 8:200-213.
Davis, K.A., K. A. Moran, C. K. McAllister, and P. J. Gray (2005) “Multidrug-resistant Acinetobacter extremity infections in soldiers” Emerging Infectious Diseases 11:1218-1224.
DeLong, E. F., G. S. Wickham, and N. R. Pace (1989) “Phylogenetic stains: ribosomal RNA-based probes for the identification of single cells” Science 243:1360-3.
DelosReyes, F. L., W. Ritter, and L. Raskin (1997) “Group-specific small-subunit rRNA hybridization probes to characterize filamentous foaming in activated sludge systems” Applied and Environmental Microbiology 63:1107-1117.
Dennis, P. P. and H. Bremer (1974) “Macromolecular-Composition during Steady-State Growth of Escherichia-Coli B-R” Journal of Bacteriology 119:270-281.
Doi, Y., S. Husain, B. A. Potoski, K. R. McCurry, and D. L. Paterson (2009) “Extensively Drug-Resistant Acinetobacter baumannii” Emerging Infectious Diseases 15:980-982.
Dupreez, J. C. and D. F. Toerien (1978) “Effect of Temperature on Growth of Acinetobacter-Calcoaceticus” Water Sa 4:10-13.
Forget, B. G. and B. Jordan (1970) “5s Rna Synthesized by Escherichia Coli in Presence of Chloramphenicol—Different 5′-Terminal Sequences” Science 167:382-&.
Goodchild, Bioconjugate Chemistry 1:165-87(1990)
Heron, M., D. L. Hoyert, S. L. Murphy, J. Xu, K. D. Kochanek, and B. Tejada-Vera (2009) “Deaths: Final Data for 2006” National Vital Statistics Reports 57.
Jones, A., D. Morgan, A. Walsh, J. Turton, D. Livermore, T. Pitt, A. Green, M. Gill, and D. Mortiboy (2006) “Importation of multidrug-resistant Acinetobacter spp infections with casualties from Iraq” Lancet Infectious Diseases 6:317-318.
Jones, K. E., N. G. Patel, M. A. Levy, A. Storeygard, D. Balk, J. L. Gittleman, and P. Daszak (2008) “Global trends in emerging infectious diseases” Nature 451:990-3.
Keller, G. H. and Manak, M. M. (1987) DNA Probes, Stockton Press, New York, NY., pp. 169-170.
Kempf, V. A. J., K. Trebesius, and I. B. Autenrieth (2000) “Fluorescent in situ hybridization allows rapid identification of microorganisms in blood cultures” Journal of Clinical Microbiology 38:830-838.
Kobayashi, G., Patrick R. Murray, Michael A. Pfaller, and Ken S. Rosenthal (2002), Medical Microbiology, published by Mosby; 4th edition.
Koch, A. L. (1971) “The adaptive responses of Escherichia coli to a feast and famine existence” Adv Microb Physiol 6:147-217.
Kollef, M. H., G. Sherman, S. Ward, and V. J. Fraser (1999) “Inadequate antimicrobial treatment of infections—A risk factor for hospital mortality among critically ill patients” Chest 115:462-474.
Leibovici, L., I. Shraga, M. Drucker, H. Konigsberger, Z. Samra, and S. D. Pitlik (1998) “The benefit of appropriate empirical antibiotic treatment in patients with bloodstream infection” Journal of Internal Medicine 244:379-386.
Levy, S. B. (1998) “The challenge of antibiotic resistance” Scientific American 278:46-53.
Licht, T. R., T. Tolker-Nielsen, K. Holmstrom, K. A. Krogfelt, and S. Molin (1999) “Inhibition of Escherichia coli precursor-16S rRNA processing by mouse intestinal contents” Environmental Microbiology 1:23-32.
MacArthur, R. D., M. Miller, T. Albertson, E. Panacek, D. Johnson, L. Teoh, and W. Barchuk (2004) “Adequacy of early empiric antibiotic treatment and survival in severe sepsis: Experience from the MONARCS trial” Clinical Infectious Diseases 38:284-288.
Maniatis et al. (1982) Molecular Cloning: A Laboratory Manual, Cold Spring Harbor Laboratory, New York.
Narang et al., Meth. Enzymol. 68:90-9 (1979)
Oerther, D. B., J. Pernthaler, A. Schramm, R. Amann, and L. Raskin (2000a) “Monitoring precursor 16S rRNAs of Acinetobacter spp. in activated sludge wastewater treatment systems” Applied and Environmental Microbiology 66:2154-2165.
Oerther, D. B., J. Pernthaler, A. Schramm, R. Amann, and L. Raskin (2000b) “Monitoring precursor 16S rRNAs of Acinetobacter spp. in activated sludge wastewater treatment systems” Appl Environ Microbiol 66:2154-65.
Pace, N. R. (1973) “Structure and Synthesis of Ribosomal Ribonucleic-Acid of Prokaryotes” Bacteriological Reviews 37:562-603.
Peleg, A. Y., Y. Tilahun, M. J. Fiandaca, E. M. C. D'Agata, L. Venkataraman, R. C. Moellering, and G. M. Eliopoulos (2009) “Utility of Peptide Nucleic Acid Fluorescence In situ Hybridization for Rapid Detection of Acinetobacter spp. and Pseudomonas aeruginosa” Journal of Clinical Microbiology 47:830-832.
Perez, F., A. M. Hujer, K. M. Hujer, B. K. Decker, P. N. Rather, and R. A. Bonomo (2007) “Global challenge of multidrug-resistant Acinetobacter baumannii” Antimicrobial Agents and Chemotherapy 51:3471-3484.
Poulsen, L. K., G. Ballard, and D. A. Stahl (1993) “Use of Ribosomal-Rna Fluorescence Insitu Hybridization for Measuring the Activity of Single Cells in Young and Established Biofilms” Applied and Environmental Microbiology 59:1354-1360.
Rosey, A. L., E. Abachin, G. Quesnes, C. Cadilhac, Z. Pejin, C. Glorion, P. Berche, and A. Ferroni (2007) “Development of a broad-range 16S rDNA real-time PCR for the diagnosis of septic arthritis in children” J Microbiol Methods 68:88-93.
Sambrook et al. (1989), Molecular Cloning, A Laboratory Manual, Second Edition, Cold Spring Harbor Press, N.Y., pp. 9.47-9.57.
Schafer, J.J. and J. E. Mangino (2008) “Multidrug-resistant Acinetobacter baumannii osteomyelitis from Iraq” Emerging Infectious Diseases 14:512-514.
Seifert, H. et al. (1993) “Antimicrobial Susceptibility of Acinetobacter Species” Antimicrobial Agents and Chemotherapy 37(4):750-753.
Sjolund, M., K. Wreiber, D. I. Andersson, M. J. Blaser, and L. Engstrand (2003) “Long-term persistence of resistant enterococcus species after antibiotics to eradicate Helicobacter pylori” Annals of Internal Medicine 139:483-487.
Smolders, G. J. F., J. Vandermeij, M. C. M. Vanloosdrecht, and J.J. Heijnen (1994) “Model of the Anaerobic Metabolism of the Biological Phosphorus Removal Process-Stoichiometry and Ph Influence” Biotechnology and Bioengineering 43:461-470.
Suggs et al. (1981) ICN-UCLA Symp. Dev. Biol. Using Purified Genes, D. D. Brown [ed.], Academic Press, New York, 23:683-693.
Waldron, C. and F. Lacroute (1975) “Effect of Growth-Rate on Amounts of Ribosomal and Transfer Ribonucleic-Acids in Yeast” Journal of Bacteriology 122:855-865.
Wisplinghoff, H. et al. (2000) “Nosocomial bloodstream infections caused by Acinetobacter species in United States hospitals: clinical features, molecular epidemiology, and antimicrobial susceptibility” Clin Infect Dis 31(3):690-7.
Woese, C. R. (1987) “Bacterial Evolution” Microbiological Reviews 51:221-271.
Zaragoza, R., A. Artero, J. J. Camarena, S. Sancho, R. Gonzalez, and J. M. Nogueira (2003) “The influence of inadequate empirical antimicrobial treatment on patients with bloodstream infections in an intensive care unit” Clinical Microbiology and Infection 9:412-418.
Zucol, F., R. A. Ammann, C. Berger, C. Aebi, M. Altwegg, F. K. Niggli, and D. Nadal (2006) “Real-time quantitative broad-range PCR assay for detection of the 16S rRNA gene followed by sequencing for species identification” J Clin Microbiol 44:2750-9.

We claim:

1. A method for determining the effect of an antimicrobial compound or composition on the specific growth rate of a target microbial population, said method comprising exposing the target microbial population to said antimicrobial compound or composition; and determining the specific growth rate of the target microbial population following exposure to said antimicrobial compound or composition.

2. The method according to claim 1, wherein said specific growth rate of the target microbial population is determined using FISH-Ribosyn.

3. The method according to claim 2, wherein said method comprises:
a) exposing a sample comprising microbes to said antimicrobial compound for a period of time;
b) exposing the sample to a protein synthesis inhibitor (e.g., chloramphenicol);
c) collecting a time series of samples (preferably at defined times) from step b; and
d) analyzing the collected samples to determine the rate of precursor rRNA accumulation in the microbes and determining the effect of said antimicrobial compound on the specific growth rate of the microbes.

4. The method according to claim 3, wherein said collected samples are analyzed in comparison to an untreated control.

5. The method according to claim 3, wherein the rate of precursor rRNA buildup/accumulation is measured in situ using fluorescence in situ hybridization (FISH).

6. The method according to claim 3, wherein said precursor rRNA is precursor 16S rRNA.

7. The method according to claim 1, wherein the target microbial population is Nitrospira spp., Nitrosospira spp., Nitrobacter spp., Nitrosomonas spp., Clostridium spp., Bacillus spp., methogenic archaea, coliforms (Enterobacteriaceae including Escherichia coli), Staphylococcus spp., Salmonella spp., Streptococcus spp., Chlamydia spp., Brucella spp., Yersinia spp., Shigella spp., Neisseria spp., Haemophilus spp., Listeria spp., Klebsiella pneumoniae, Pseudomonas spp., Mycobacterium spp., Bordetella spp., Actinomycetes spp., Vibrionaceae spp., Treponema spp., Legionella spp., Mycoplasma spp., Rickettsiae spp., or Bacteroides spp.

8. The method of claim 3, wherein the at least one protein synthesis inhibitor is chloramphenicol, lincomycin, or erythromycin.

9. The method of claim 3, wherein said determining comprises contacting the samples with a labeled hybridization probe targeting the precursor 16S rRNA of the microbial population, and detecting a signal from the probe, wherein the signal is indicative of the number of ribosomes present in each sample.

10. The method of claim 9, wherein the probe targets the 5′ end or 3′ end of precursor 16S rRNA.

11. The method of claim 9, wherein the probe targets the interior region of both precursor 16S rRNA and mature 16S rRNA.

12. The method of claim 3, wherein said determining comprising carrying out fluorescence in situ hybridization (FISH) with an oligonucleotide probe targeting the precursor 16S rRNA of the microbial population.

13. The method of claim 12, wherein the probe targets the 5′ or 3′ end of precursor 16S rRNA.

14. The method of claim 12, wherein the probe targets the interior region of both ‘ precursor 16S rRNA and mature 16S rRNA.

15. The method of claim 3, wherein said determining comprises contacting the samples with primers targeting the precursor 16S rRNA of the microbial population, wherein an amplification product is indicative of the number of ribosomes present in each sample.

16. The method of claim 3, further comprising inputting the rate of pre16S rRNA accumulation of the microbial population into a computer algorithm that calculates the specific rate of ribosome synthesis.

17. The method of claim 3, further comprising recording the specific growth rate or specific rate of ribosome synthesis of the microbial population in physical or electronic media.

18. The method of claim 3, further comprising comparing the specific growth rate of the microbial population with that of a known reference microbial population.

19. A kit for determining the specific growth rate of a microbial population, comprising packaging; and a compartment containing one or more oligonucleotide probes or primers that target sequence within the precursor 16S rRNA and/or mature 16S rRNA; and one or more compartments containing one or more antibiotics.

20. The kit of claim 19, wherein the probe or primers target the 5’ or 3′ end of precursor 16S rRNA, or the interior region of both precursor 16S rRNA and mature 16S rRNA.

21. The kit of claim 19, further comprising at least one component selected from the group consisting of a protein synthesis inhibitor, a reagent to conduct an amplification reaction, means for obtaining a biological or environmental sample, and a set of instructions relating information regarding components of the kit and/or how to measure specific growth rate of a microbe.


SEQUENCE LISTING (1 sequence)

SEQ ID NO: 1 - DNA, 24 bp, artificial sequence
  oligonucleotide hybridization probe
gattcttaccaatcgtcaatcttt